(19) United States
(12) Patent Application Publication
(10) Pub. No.: US 2011/0177059 A1
(43) Pub. Date: Jul. 21, 2011

(54) PSA AND KLK2 AS THERAPEUTIC TARGETS AND MOLECULES INHIBITING PSA AND KLK2

(75) Inventor: Chawnshang Chang, Pittsford, NY (US)

(73) Assignee: UNIVERSITY OF ROCHESTER, Rochester, NY (US)

(21) Appl. No.: 13/057,626

(22) PCT Filed: Aug. 5, 2009
(86) PCT No.: PCT/US09/52779

§ 371 Date: Apr. 6, 2011

Related U.S. Application Data
(60) Provisional application No. 61/086,461, filed on Aug. 5, 2008.
(60) Provisional application No. 61/093,285, filed on Aug. 29, 2008.
(60) Provisional application No. 61/093,353, filed on Aug. 31, 2008.

(51) Int. Cl. A61K 39/395; G01N 33/566; G01N 33/53

(52) U.S. Cl. 424/130.1; 436/501; 435/7.1

(57) ABSTRACT

Disclosed herein are compositions and methods relating to prostate-specific antigen (PSA), KLK2 and androgen receptor. Further provided are methods and compositions for inhibiting PSA and/or KLK2 activity. Further provided are compositions and methods for treating or preventing cancer.

This application claims priority to U.S. Application No. 61/086,461, filed Aug. 5, 2008, U.S. Application No. 61/093,285, filed Aug. 29, 2008, and U.S. Application No. 61/093,353, filed Aug. 31, 2008. U.S. Application No: 61/086,461, filed Aug. 5, 2008, U.S. Application No. 61/093,285, filed Aug. 29, 2008, and U.S. Application No. 61/093,353, filed Aug. 31, 2008 are hereby incorporated herein by reference in their entirety.

### I. ACKNOWLEDGEMENTS

This invention was made with government support under federal grants DK093414, DK60912 and CA122295 awarded by the National Institutes of Health and the George H. Whipple Professorship Endowment. The Government has certain rights to this invention.

### II. BACKGROUND

Prostate-specific antigen (PSA), a member of the kallikrein gene family is a serine protease with chymotrypsin-like activity that is expressed mainly in prostate¹. Evidence indicates that PSA, as well as other tissue kallikrein members involved in tumorgenesis primarily in hormone-related malignancies², including ovarian carcinoma³, breast cancer⁴, prostate cancer, lung adenocarcinoma⁵, pancreatic ductal adenocarcinomas⁶ and lymphblastic leukemia⁷. PSA has been known as the best biomarker for monitoring prostate cancer progression⁸. In prostate tissue, PSA expression levels are correlated with clinical stage and cytological grade⁹. In localized prostate tumor, T3 stage tumors produced higher tissue PSA than T2 stage tumors, while metastatic tumors have relatively low PSA expression⁹. Early data suggested that PSA might promote prostate cancer growth and metastasis via its protease activity to digest IGFBP3¹⁰ or hydrolyze several extracellular matrixes¹¹.

Androgen receptor (AR) is the primary regulator of PSA expression through its three androgen response elements (AREs) located in the proximal 6 Kb of the PSA promotor¹². PC-3 cells with stably-transfected AR, but not parental PC-3 cells lacking AR, could express PSA¹³. Therefore, during the hormone treatment sensitive stage, surgical or medical castration can significantly reduce PSA expression in prostate cancer tissue. But in the hormone-refractory cancer, although AR still exists¹⁴, the abnormal PSA elevation may not attribute to AR regulation in this stage. It has been reported that in addition to androgens, PSA expression may be induced by glucocorticoids¹⁵, progestin¹⁶, and Ets transcription factors¹⁷. However, the significance of AR-independent PSA expression remains unclear. It is disclosed herein that PSA can promote hormone refractory prostate cancer growth via enhancing ARA70-induced AR transactivation without involving its protease activity. Under the treatment of hydroxyflutamide (IIF) or in the environment of Delta 5-androstenediol (Adiol), the increase tissue PSA can activate ARA70/AR transcription function, which can then result in tumor cell survival in the hormone refractory tissue. Therefore, target tissue PSA by PSA-siRNA or smaller molecules represent therapeutics to suppress prostate cancer growth.

### III. SUMMARY

In accordance with the purposes of this invention, as embodied and broadly described herein, this invention, in one aspect, relates to compositions and methods related to inhibiting PSA to inhibit cancer.

It is to be understood that both the foregoing general description and the following detailed description are exemplary and explanatory only and are not restrictive of the invention, as claimed.

### IV. BRIEF DESCRIPTION OF THE FIGURES

FIG. 1 shows the growth of human prostate tumors is correlated with their tissue PSA levels. Histological analyses of the cell proliferation marker Ki67, cell apoptosis (TUNEL), and the tissue expressions of PSA, AR, and ARA70 in non-hormone treatment, HF treatment sensitive and refractory prostate tumor specimens. (a-d) Immunohistochemical staining for Ki67 shows lower levels of cell proliferation in HF sensitive (b) and higher levels of cell proliferation in HIT refractory prostate cancer specimens (c) as compared with that in non hormone treatment specimens (a), as demonstrated by counting the percentage of positive staining cells in 5 separate fields on each specimen with results from the average of a total of 30 fields (d). (e-h) TUNEL assay shows that the cell apoptosis signals in HF sensitive tumors (f) are higher than those in non-hormone treatment tumors (e) and refractory tumors (g). (i-l) IHC staining shows tissue PSA levels are higher in hormone refractory tumors (k) and lower in HF sensitive tumors (j) as compared with those in non-hormone treatment tumors (i). The positive staining is semi-quantitated by Image J software (l). The increased tissue PSA levels in the hormone refractory tumors are correlated with higher cell proliferation and lower apoptosis index in those samples (P<0.01, data not show).

FIG. 2 shows PSA accelerated cell growth in AR-positive, but not in AR-negative prostate cancer cells. Two AR-positive hormone refractory models, high passage LNCaP and CWR22rv1 cells, and two AR-negative hormone refractory models, PC-3 and Du-145 cells, are stably transfected with pcDNA3-PSA. Cell viability is determined by MTT assay, which is interpreted into cell cycle and cell death analysis. (a-c) Stably over-expression of PSA promotes cell growth of high passage LNCaP cells (LN-PSA) with 1 nM DHT treatment using MTT assay (a). Flow cytometry analysis of cell cycle by detecting propidium iodide staining reveals that the higher expression of PSA in LN-PSA cells resulted in the decreased GO-G1 phase from 69% to 54% and increased S phase from 18% to 29% (b). Flow cytometry analysis of cell apoptosis by detecting 7-AAD staining shows less apoptotic cells in LN-PSA cells than control LN-vector cells (c). (d4) PSA has similar effects on AR-positive CWR22rv1 cell as on high passage LNCaP cells. (g-l) Over-expression of PSA in PC-3 cells (PC3-PSA) does not show the change of cell growth and apoptosis by compared with parental PC-3 cells transfected with pcDNA3 vector. The same conclusion was drawn in PSA over-expression Du145 cells (Du-PSA) (j-l).

FIG. 3 shows siRNA knockdown of PSA reduces cell growth in AR-positive LNCaP cells and CWR22rv1 cells. (a) Western blotting shows PSA expression has been knocked down in high passage LNCaP cells, (LN-siPSA clone 1, 2, 3, and 4) using siRNA strategy. (b-d) Knockdown of endogenous PSA via PSA-siRNA reduces growth rates of high passage LNCaP cells treated with 1 nM DHT using MTT assay (b), results in the increased G0-G1 phase from 61% to 79% and decreased S phase from 25% to 11% (c), and induces apoptosis (d). (e-h) Knockdown endogenous PSA via PSA-siRNA in CWR22rv1 cells (CWR-siPSA clone 1, 2, 3 and 4) reduces growth rates in MTT assay (f), results in the increased G0-G1 phase and decreased S phase (g), and induces apoptosis (h).

FIG. 4 shows PSA cooperates with ARA70 to enhance AR transactivation. (a) PSA enhances AR transactivation and PSA-siRNA inhibits AR transactivation in stably transfected LNCaP cells. MMTV-luciferase activity was measured when PSA was over-expressed or knockdown in LNCaP cells. Using the LNCaP stable cell lines, AR transactivation was effectively suppressed by PSA-siRNA and ARA70-siRNA, and further enhanced by over-expression of PSA. (b) PSA enhances the expression of AR target genes, PSP94, PSMA, and NKX3.1, in LNCaP cells in real-time PCR assay. LN-PSA versus LN-vector: **P<0.01; LN-siPSA versus LN-scramble: *P<0.05. (c) Over-expression of PSA enhances AR transactivation and knockdown of PSA or ARA70 inhibits AR transactivation in CWR22rv1 cells. Using the CWR22rv1 stable cell lines, AR transactivation on MMTV-ARE luciferase reporter (MMTV-Luc) was suppressed by PSA-siRNA and ARA70-siRNA, and could be enhanced by over-expression of PSA. (d) PSA and ARA70 collaboratively enhance HF-, or Adiol-mediated AR transactivation. COS-1 cells were transfected with MMTV-Luc and pSG5-AR in the presence or absence of pSG5-ARA70F or pCDNA3-PSA. The co-transfection of ARA70 and PSA further trigger the 10 uM HF- or 10 nM Adiol-induced AR transactivation.

FIG. 5 shows PSA protease activity is not critical for its effects on cell growth and AR transactivation in prostate cancer cells. (a) PSA overexpressed high passage LNCaP cells (LN-PSA) and control LN-vector cells were treated with PSA proteinase inhibitor, a₁,-antichymotrypsin (ACT, 1000 ng/ml), for the indicated time courses. The growth of enzyme inhibitor treated cells was not significantly changed compared with the vehicle (1× PBS) treated cells. (b) Both Wt PSA and enzyme activity-null mutant PSA function as growth stimulators in high passage LNCaP cells. Wt and mutant PSA were stably introduced into LNCaP cells, LN-PSA and LN-mPSA, respectively, and the cell growth rates were determined by MTT assay. (c) The increase of secreted PSA follows the increase of cellular PSA after DHT treatment in LNCaP cells. Cells were cultured in medium with charcol-stripped serum for 24 h, and stimulated with 10 nM DHT. Cell lysate and conditioned medium were harvested as indicated time points. (d) The secreted PSA does not further enhance the growth of prostate cancer cells. The 48 hr culture medium from LN-PSA cells was collected and used as the conditioned medium to grow LNCaP cells. The proliferation rate was compared with that of cells grown under normal medium. Cells were then collected and proliferating rates were examined using MTT assay. (e-f) MMTV-ARE lucferase assays show both Wt PSA and enzyme activity-null PSA can cooperate with ARA70 to enhance AR transactivation in Cos-1 cells (e) and high passage LNCaP cells (1).

FIG. 6 shows PSA/ARA70/AR may modulate prostate cancer cell death and proliferation via regulating p53 and cdk2/cyclin D1 expression. (a) The differences of the apoptosis signal between LN-PSA and LN-vector are further magnified by treatment of 1 nM TPA. Following 24 hr challenge with 1 nM TPA, the expression of apoptosis associated proteins, bcl-2, bax, total and active forms of p53 (phospho-p53 scr392), in LN-PSA and LN-Vector cells are analyzed by Western blotting. Two caspase-3 activate subunits, 17 kDa and 12 kDa, are also measured. Over-expression of PSA in LN-PSA cells significantly reduces apoptosis induced by 1 nM TPA, via decreasing total p53 expression. The consequence of reduced total p53 expression may result in decreased phospho-p53 levels, elevated bcl2/bax ratio and diminished active caspase-3 in LN-PSA cells (lower panel). (b) PSA expression status affects the expression levels of cell cycle proteins (p21, cdk2, and cyclinD1) and cell proliferation markers (PCNA, and RFCI) in AR positive prostate cancer cells. Western blots analyses shows that over-expression of PSA in high passage LNCaP cells results in higher expression of p21, cdk2, cyclinD1, ORC1, and RFC1, whereas knockdown of PSA has opposite effects on the markers. (c) PSA affects the cell viability of prostate cancer cells via Colony Formation assay of high passage LNCaP cells. The spot with cell numbers higher than 30 is counted as one colony. (d) PSA expression level is positively correlated with the tumorigenicity of prostate cancer xenografts. The high passage LNCaP cells with higher levels of PSA generate larger size xenograft tumors than LN-siPSA cells. Equal numbers of high passage LNCaP and LN-siPSA cells were mixed with Matrigel and then inoculated into the left and right flanks of pre-castrated athymic nude mice, respectively. Tumors were harvested and weighted 12 weeks after the xenograft implantation.

FIG. 7 shows KLK2 increases cell growth in AR-positive LNCaP cells, but not in AR-negative PC-3 cells. (A) KLK2, ARA70 and AR are differentially expressed in various cell lines. (B) Cell growth was measured by MTT assay on LNCaP and PC-3 stably transfected cell lines treated with 10 nM DHT. (C) Flow cytometry analysis of cell cycles by measuring DNA amounts in each cell. (D) Cell apoptosis was detected by flow cytometry of 7-AAD staining.

FIG. 8 shows KLK2 enhances ARA70-induced AR transactivation. (A) KLK2 enhances 1 nM DHT and 10 nM DHT mediated induction of AR transcriptional activity by cooperation with ARA70 in MMTV-Luc and PSA-Luc assay in COS-1 cells. (B and C) KLK2 enhances 10 ?μM hydroxyflutamine (HF) and 10 nM estradiol (E2) mediated induction of AR transcriptional activity in MMTV-Luc and PSA-Luc assays.

FIG. 9 shows ARA70 mediates KLK2 enhancement function. (A) AR transactivation, measured by MMTV-luciferase activity, was increased by KLK2 in LNCaP cells. (B) The transactivity of AR, which was transfected into PC-3 cells, was induced by ARA70 and KLK2. (C) AR transactivation was suppressed by KLK2 siRNA and ARA70 siRNA (D) in LNCaP cells.

FIG. 10 shows using stably transfected LNCaP cell lines, AR target gene expression was modulated by KLK2. (A) the KLK2 expression levels were detected by RT-PCR and Real-time PCR in stably subline LN-vector, LN-KLK2, and LN-siKLK2. (B) Real-time PCR and Western blot analysis of the expression of AR target gene, PSP94 and NKX3.1 in LN-vector, LN-KLK2 and LN-siKLK2 sublines.

FIG. 11 shows KLK2 functions as modulator to enhance ARA70-induced AR transactivation. (A) KLK2 and ARA70 interact in vitro by Mammalian Two-Hybrid method, comparing with other AR coactivators, such as ARA54, ARA55 and SRC-1 (B). (C) GST pull-down assays were carried out to show that radiolabeled KLK2 protein could interact with the GST-ARA70 fusion proteins.

FIG. 12 shows KLK2 enhanced ARA70-induced AR transactivation might result in the increased cell growth via modulation of MDM2 which targets p53 mediated cell growth arrest and apoptosis. (A) Western blots were carried out to measure the expression of MDM2, p53, p21, cdk2, bcl-2, and bax in LN-KLK2, LN-siKLK2, and control cell lines. (B) The sketch to show the mechanism of KLK2 function.

FIG. 13 The interaction of PSA and ARA70. (a) Interaction of PSA and ARA70 using GST pull-down assays. GST PSA recombinant protein is incubated with [³⁵S]-methionine labeled ARA70, and the protein complex is precipitated by anti-GST antibody. Our results indicate the GST PSA could interact with ARA70 with GST used as negative control. (b) Co-precipitation of endogenous ARA70, PSA and AR in high passage LNCaP cells. Using anti-ARA70 monoclonal antibody, we precipitated the ARA70 binding protein complex from 1000 μg LNCaP protein lysate. The results indicated that PSA-ARA70-AR complex exists in prostate cancer cells. (c) ARA70 and PSA could be colocalized in the cytosol of high passage LNCaP cells treated with 1 nM DHT on confocal microscope. PSA, ARA70 and AR are firstly recognized by goat anti-PSA, mouse anti-ARA70 and rabbit anti-Giantin antibody, and secondly developed into green (Alexa Fluors 488), red (TEXAS-RED) and blue (Alexa Fluors 647) fluorescence. PSA and ARA70 mainly existed in the cytosol, while AR could be located in both cytosol and nuclear in the high passage LNCaP cell treated by 1 nM DHT. When overlapping PSA green color to the ARA70 red color in Merge 1, it shows that PSA diffused in cytosol and colocalize with ARA70. Merge 2 shows that PSA colocalized with ARA70 and AR in the cytosol. (d) PSA (green color) is not restricted inside of the golgi's apparatus (blue) using confocal microscope.

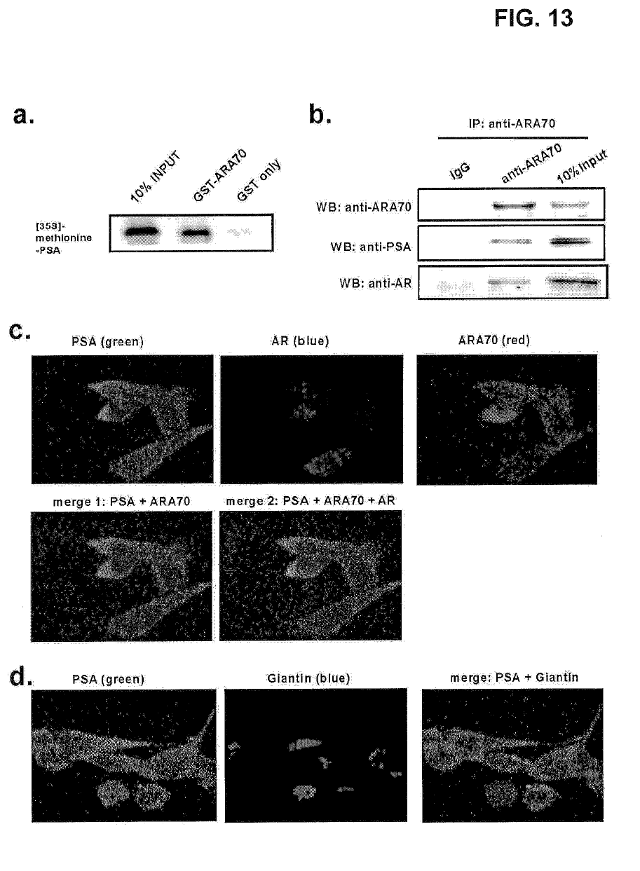

FIG. 14(a). The endogenous expression levels of AR, PSA and ARA70 in LNCaP, CWR22rv1, PC-3 and COS-1 were examined using Western blotting assay. (b) LNCaP, CWR22rv1, PC-3 and Du145 cells were transfected with pCDNA3-vector or pCDNA3-PSA. The expression of PSA in these stable cell clones was determined by Western Blot assay. (c) PSA alone, without ARA70, could not enhance AR transactivation using MMTV-luciferase assay in COS-1 cells (lane 7 vs lane 2). (d) The knockdown efficiency of ARA70siRNA, compared with its scramble siRNA, was demonstrated by assaying ARA70 protein levels.

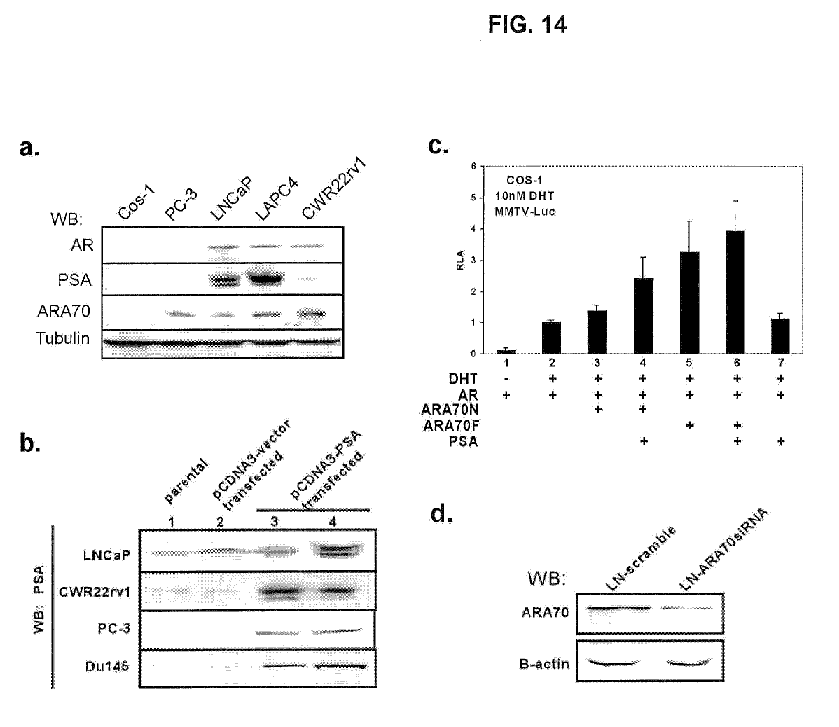

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art, which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Coapplication” is defined as the application of one or more substances simultaneously, such as in the same formulation or consecutively, within a time frame such that each substance is active during a point when the other substance or substances are active.

The terms “higher,” “increases,” “elevates,” or “elevation” or variants of these terms, refer to increases above basal levels, e.g., as compared a control. The terms “low,” “lower,” “reduces,” “decreases” or “reduction” or variation of these terms, refer to decreases below basal levels, e.g., as compared to a control. For example, basal levels are normal in vivo levels prior to, or in the absence of or addition of an agent such as an agonist or antagonist to activity. For example, decreases or increases can be used to describe a PSA interaction with ARA70 or a KLK2 interaction with ARA70. In this context, descreases would describe a situation of where the interaction of PSA with ARA70 was defined as having a Kd of 10⁻⁹ M, if this intraction decreased, meaning the binding lessened, the Kd could decrease to 10⁻⁶ M. It is understood that wherever one of these words is used it is also disclosed that it could be 1%, 5%, 10%, 20%, 50%, 100%, 500%, or 1000% increased or decreased from a control.

Inhibit or forms of inhibit refers to to reducing or suppressing. Thus, inhibiting the activity of PSA or inhibiting a PSA-ARA70 interaction means reducing or suppressing the same respectively.

The term modulate refers to its standard meaning of increasing or decreasing. It is understood that wherever one of these words is used it is also disclosed that it could be 1%, 5%, 10%, 20%, 50%, 100%, 500%, or 1000% increased or decreased from a control.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control. It is understood that wherever one of these words is used it is also disclosed that it could be 1%, 5%, 10%, 20%, 50%, 100%, 500%, or 1000% increased or decreased from a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test compound on a cell was in question, one could a) simply record the characteristics of the cell in the presence of the compound, b) perform a and then also record the effects of adding a control compound with a known activity or lack of activity and then compare effects of the test compound to the control compound. In certain circumstances once a control is performed the control can be used as a standard, in which the control experiment does not have to be performed again and in other circumstances the control experiement should be run in parallel each time a comparison will be made.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

There are many different types of cells disclosed, including prostate cells and prostate cancer cells. Prostate cancer can be hormone refractory prostate cancer (HRPC) which can be a synonym for androgen-independent prostate cancer (AIPC). A definition of HRPC is prostate cancer that has become refractory, that is, it no longer responds to hormone therapy. A definition of AIPC is a prostate cancer that does not require androgen to progress. A HRPC cell or an AIPC cell refers to a cell having properties characteristic of HRPC or AIPC.

When used with respect to pharmaceutical compositions, the term “stable” is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

It is understood that wherever the word cancer appears without prostate as a modifier, it is,understood that prostate cancer is also disclosed.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Throughout this application, various publications are referenced. The disclosures of these publications in their entireties arc hereby incorporated by reference into this application in order to more fully describe the state of the art to which this pertains. The references disclosed arc also individually and specifically incorporated by reference herein for the material contained in them that is discussed in the sentence in which the reference is relied upon.

A system as used herein refers to an interdependent group of items forming a unified whole. For example, a computer system are the parts, such as a process, a memory storage device, and other parts which can be used to form a functioning computer. Also for example, a system can be made up of a cell and the necessary reagents for the cell to be passaged. Also by example, the word system can be used as a system, wherein the system comprises PSA and ARA70, and wherein a determination can be made as to whether PSA interacts with ASA70. In this type of system, for example, the system could be a test tube, having the necessary reagents to determined if PSA interacts with ARA70, it could be a column chromatography system, a batch binding sytem, a immobilized bead system, a free bead system etc. The system could also be a cell based system, wherein the cell expresses the necessary genes and is in the necessary conditions to read out an assay which is predictive of PSA interacting with ARA70. Those of skill in the art, given the information herein, can create systems around particular pieces of information, once the information is provided, such as information about an interaction between two proteins.

A number of interactions are referred to herein, such as a PSA interaction with ARA70 and a KLK2 interaction with ARA70, an inhibitor interaction with PSA, KLK2, or ARA70, or a test compound interaction with PSA, KLK2, or PSA. Interaction, interacting, or interact or other forms of the word as used in this type of context refers to when the two molecules touch in a measurable way, i.e. bind. Typcially binding is refered to as binding constants, and where interactions are discussed, it is understood that the interaction can be limited based on the strength of the interaction as measured by a dissociation constant, Kd. A Kd typically is quantified by M, for example, 10⁻¹⁴ M, 10⁻¹³ M, 10⁻¹² M, 10⁻¹¹ M, 10⁻¹⁰ M, 10⁻⁹ M, 10⁻⁸ M, 10⁻⁷M, 10⁻⁶M, 10⁻⁵ M, 10⁻⁴ M, 10⁻³ M. The Kd can be used to describe the affinity between a ligand (L) (such as a drug) and a protein (P) i.e. how tightly a ligand binds to a particular protein, such as a PSA inhibitor and PSA or ARA70. It is understood, that in some circumstances, the ligand can be a protein as well, such as in describing a PSA and AR70 interaction. Ligand-protein affinities are influenced by non-covalent intermolecular interactions between the two molecules such as hydrogen bonding, electrostatic interactions, hydrophobic and Van der Waals forces.

PSA activity refers to the activities disclosed herein, such as the activities of cell proliferation, ARA70 binding, and AR transactivation and downstream transactivation. Also disclosed are protease independent PSA activities, which refers to activities not dependent on the protease activity of PSA.

KLK2 activity refers to the activities disclosed herein, such as the activities of cell proliferation, ARA70 binding, and AR transactivation and downstream transactivation. Also disclosed are protease independent KLK2 activities, which refers to activities not dependent on the protease activity of KLK2.

A subject would be in need of a PSA inhibitor, a KLK2 inhibitor, or other inhibitors as disclosed herein, at least if the subject has prostate cancer, has a need for decreasing prostate cancer cell proliferation, or has HRPC or AIPC. It is understood that monitoring a subject, by for example, determining whether there is a need for a PSA inhibitor, a KLK2 inhibitor, or other inhibitors as disclosed herein, after some form of treatment has occurred, for example, prostate cancer treatment, including administration of a PSA inhibitor, a KLK2 inhibitor, or other inhibitor disclosed herein.

As used herein, “mimic” refers to performing one or more of the functions of a reference object. For example, an ARA70 mimic performs one or more of the functions of ARA70.

### B. COMPOSITIONS AND METHODS

### 1. Prostate Cancer—General

Prostate cancer is a disease in which cancer develops in the prostate, a gland in the male reproductive system. It occurs when cells of the prostate mutate and begin to multiply out of control. These cells may spread (metastasize) from the prostate to other parts of the body, especially the bones and lymph nodes. Prostate cancer may cause pain, difficulty in urinating, erectile dysfunction and other symptoms.

Rates of prostate cancer vary widely across the world. Although the rates vary widely between countries, it is least common in South and East Asia, more common in Europe, and most common in the United States. According to the American Cancer Society, prostate cancer is least common among Asian men and most common among black men, with figures for white men in-between. However, these high rates may be affected by increasing rates of detection.

Prostate cancer develops most frequently in men over fifty: This cancer can occur only in men, as the prostate is exclusively of the male reproductive tract. It is the most common type of cancer in men in the United States, where it is responsible for more male deaths than any other cancer, except lung cancer.

Prostate cancer can be divided into 4 stages (classified I-IV). Stage I disease is cancer that is found incidentally in a small part of the sample when prostate tissue was removed for other reasons, such as benign prostatic hypertrophy, and the cells closely resemble normal cells and the gland feels normal to the examining finger. In Stage It more of the prostate is involved and a lump can be felt within the gland. In Stage III, the tumor has spread through the prostatic capsule and the lump can be felt on the surface of the gland. In Stage IV disease, the tumor has invaded nearby structures, or has spread to lymph nodes or other organs. Grading is based on cellular content and tissue architecture from biopsies (Gleason) which provides an estimate of the destructive potential and ultimate prognosis of the disease.

### a) Prostate Cancer Cell Lines

There are a variety of prostate cancer cell lines which have been derived from prostate cancer cells and have one or more properties of a prostate cancer cell.

### (1) LNCaP Cell Line

LNCap cells are a cell line of human cells commonly used in the field of oncology. LNCaP cells are androgen-sensitive human prostate adenocarcinoma cells derived from the left supraclavicular lymph node metastasis from a 50-year-old caucasian male in 1977. They are adherent epithelial cells growing in aggregates and as single cells.

The LNCaP cell line was established from a metastatic lesion of human prostatic adenocarcinoma. The LNCaP cells grow readily in vitro (up to 8×105 cells/sq cm; doubling time, 60 hr), form clones in semisolid media, are highly resistant to human fibroblast interferon, and show an aneuploid (modal number, 76 to 91) human male karyotype with several marker chromosomes. The malignant properties of LNCaP cells are maintained. Athymic nude mice develop tumors at the injection site (volume-doubling time, 86 hr). Functional differentiation is preserved; both cultures and tumor produce acid phosphatase.

High-affinity specific androgen receptors are present in the cytosol and nuclear fractions of cells in culture and in tumors. Estrogen receptors are demonstrable in the cytosol. The model is hormonally responsive. In vitro, 5 alpha-dihydrotestosterone modulates cell growth and stimulates acid phosphatase production. The cell line does express PSA (Prostate Specific Antigen). In vivo, the frequency of tumor development and the mean time of tumor appearance are significantly different for either sex. Male mice develop tumors earlier and at a greater frequency than do females. Hormonal manipulations show that, regardless of sex, the frequency of tumor development correlates with serum androgen levels. The rate of the tumor growth, however, is independent of the gender or hormonal status of the host.

### (2) C4-2 Cell Line

C4-2 cells are androgen-independent and highly tumorigenic and have a proclivity to metastasize to the bone. It is believed that changes in nuclear DNA or mitochondrial DNA (mtDNA) induced by androgen ablation altered the status of androgen-dependent LNCaP to create the androgen-independent C4-2.

### (3) DU145 Cell Line

The DU145 long-term tissue prostate cancer cell line was derived from brain metastasis in a 69-year-old Caucasian male with prostate carcinoma in 1975. DU145 is androgen-independent. DU145 are not hormone sensitive and don't express PSA (Prostate Specific Antigen). The cells are epithelial and grown in isolated islands on plastic Petri dishes, and form colonies in soft agar suspension culture. Karyotypic analysis demonstrates an aneuploid human karyotype with a modal chromosome number of 64. It is a hypotriploid human cell line. Both 61 and 62 chromosome numbers had the highest rate of occurrence in 30 metaphase counts. The rate of higher ploidies was 3%. The t(11q12q), del(11)(q23), 16q+, del(9)(p11), del(1)(p32) and 6 other marker chromosomes were found in most cells. The N13 was usually absent. The Y chromosome is abnormal through translocation to an unidentified chromosomal segment. The X chromosome was present in single copy. The line is not detectably hormone sensitive, is only . weakly positive for acid phosphatase and isolated cells form colonies in soft agar. The cells do not express prostate antigen. Ultrastructural analyses of both the cell line and original tumor revealed microvilli, tonofilaments, desmosomes, any mitochondria, well developed Golgi and heterogenous lysosomes.

### (4) PC-3 Cell Line

The PC-3 was initiated from a bone metastasis of a grade IV prostatic adenocarcinoma from a 62-year-old male Caucasian. PC-3 is androgen-independent. The cells exhibit low acid phosphatase and testosterone-5-alpha reductase activities. The line is near-triploid with a modal number of 62 chromosomes. There are nearly 20 marker chromosomes commonly found in each cell; and normal N2, N3, N4, N5, N12, and N15 are not found. No normal Y chromosomes could be detected by Q-band analysis.

### (5) PC-3M Cell Line

PC-3M is a highly invasive metastatic human prostate cancer cell line. The cell line is androgen-independent.

### 2. PSA and PSA as a Target

Disclosed herein is data that shows that PSA, not only is a marker for cancer, such as prostate cancer, but it is also a therapeutic target for the treatment of cancer, such as prostate cancer. PSA is a protease, and it has been shown that inhibiting the protease activity is not acceptable as a treatment for prostate cancer. Disclosed herein, the cancer proliferating activity of PSA is independent of its protease activity and can be promoted through an interaction with an Androgen receptor activator, ARA70. Through this interaction with ARA70, PSA can promote cancer cell growth, such as prostate cancer cell growth and proliferation.

Disclosed are methods of identifying PSA inhibitors which inhibit the cancer proliferation activity of PSA, such as the activity promoted by the PSA-ARA70 interaction. Also disclosed are methods of identifying cancers which are promoted by PSA. In addition, disclosed are methods of treating cancer and compositions that are PSA inhibitors that can be used, for example as in methods of treating cancer, such as prostate cancer.

Disclosed herein tissue PSA is involved in the development of hormone-refractory prostate cancer. This means, that not only does inhibition of PSA reduce cancer, such as prostate cancer proliferation, inhibition of PSA also reduces cancer cell, such as prostate cancer proliferation in cells which are no longer responsive in a negative way to, for example, hydroxyl flutamide (HF). Thus, inhibition of PSA cancer promoting activity can act to decrease cell proliferation of cancer, such as prostate cancer, which is no longer responsive to traditional ablation therapy. Consistent with this, histological analyses show the increased tissue PSA levels are correlated with lower cell apoptosis index and higher cell proliferation rate in hormone-refractory tumors specimens. Also consistent with this, PSA was found to promote the growth of AR-positive CWR22rvl and high passage LNCaP (hormone refractory prostate cancer cells), but not that of AR-negative PC-3 and DU145 cells.

PSA can induce AR transactivation via cooperating with ARA70. This can result in decreased apoptosis and increased cell proliferation in AR positive, yet hormone insensitive prostate cancer cells.

Also disclosed herein, the protease activity of PSA is not crucial for PSA to stimulate growth and promote AR transactivaton.

One way of inhibiting PSA cancer cell proliferation activity is through the knockdown of PSA in a cancer cell, such as a prostate cancer cell. In this context, knockdown of PSA refers to functional nucleic acid activities, such as siRNA, RNAi, antisense, or even ribozymes, for example, which will reduce the amount of PSA transcript in a cancer cell, such as a prostate cancer cell. If inhibition of PSA cancer cell proliferation activity occurs in cancer cells, such as in LNCaP and CWR22rv1 cells this causes cell apoptosis and cell growth arrest at the GI phase. Targeting PSA for inhibition results in the suppression of cancer growth, such as prostate cancer growth.

Disclosed are PSA inhibitors, such as PSA inhibitors that inhibit the cancer cell proliferation activity, such as prostate cancer cell proliferation activity. A PSA inhibitor can be any compound that reduces any PSA activity as defined herein, such as binding ARA70 or decreasing AR target gene activation or decreasing cell proliferating activity. In certain embodiments the PSA inhibitor is a PSA inhibitor that does not directly interact with the protease site and/or the protease activity. In certain embodiments, a PSA inhibitor is a competitive inhibitor for ARA70 interaction. Thus, disclosed are PSA inhibitors which bind PSA and competitively compete with ARA70 binding.

A PSA inhibitor can also be an antibody, protein, peptide, amino acid, or derivative or mimetic, a functional nucleic acid, such as a siRNA. For example, a functional nucleic acid PSA inhibitor can be to the following target sites for PSA, PSA-siRNA can be the target sites for PSA GTGGATCAAGGACACCATC (753-771). An ARA inhibitor can be an siRNA that targets the following site in ARA70 and GAGGAGACACTTCAACAGC (384-402).

Also disclosed are ARA70-PSA inhibitors, which are molecules that function as competitive inhibitors for PSA binding. Thus, disclosed are ARA70-PSA inhibitors that bind ARA70 and competitive inhibit PSA interaction with ARA70.

Disclosed herein, the accelerated growth of the hormone refractory prostate tumor is correlated with the increased tissue PSA expression. PSA increases cell growth in AR-positive LNCaP and CWR22rv1 cells, but not in AR-negative PC-3 and Du145 cells. Disclosed are diagnostic and prognostic assays for a subject comprising assaying PSA and correlating the amount to a control or a standard for prediction about the effects of hormone refractory stage prostate cancer.

Also disclosed are results that revealed that the higher expression of PSA in human prostate refractory tumors facilitates tumor cell survival even during HF treatment by resistant to cell death and accelerating cell cycle.

Disclose are methods of treatment of prostate cancer wherein the inhibition of PSA cancer cell proliferation activity is performed in cancers that are AR positive, for presence and/or activity.

Disclosed herein, PSA enhances AR transactivation via interaction with ARA70.

Also disclosed herein, inhibition of PSA inhibits AR positive regulated genes, such as PSP94²³ and Nkx3.1²⁴. Assaying these genes for the amount of PSA activity, wherein the activity of PSA increases AR activity in a cancer cell, such as cancer cell proliferation activity, such as in a prostate cancer cell. Likewise, inhibition of PSA increases endogenous AR negative-regulated target genes such as PSMA^{25,26} with the concomitant assays for PSA inhibition activity.

In certain assays, high passage, and or stably transfected, LNCaP cells can be used and can be grown in RPMI-1640 (Life Technologies, Rockville, Md.) with 10% CD serum, 100 units/ml of penicillin, and 100 ug/ml streptomycin under 5% CO2. 5a-Dihydrotestosterone (DHT) and delta5-androstenediol (Adiol) were obtained from Sigma and hydroxyflutamide (HF) was from Schering.

In certain assays, COS-1, PC-3, LNCaP and CWR22rv1 cells can also be used, and can be grown in appropriate medium at 1-4×10⁵ cells in 24-well plates, were transfected with indicated plasmids using SuperFect (Qiagen) according to the manufacturer's procedure. After incubation for 2-3 hr, the medium was changed and cells were treated with ethanol, DHT, or other ligands for 24 hr. After washing with 1× PBS twice, the cells were harvested in 100 μl of passive cell lysis buffer (Promega) at 4° C. for 20 mins. The luciferase activity in 20 μl cell lysate was measured by the Dual-Luceiferase Reporter 1000 Assay system (Luminometer, Tunner Designs) with MMTV-Luc as reporter of AR and pRL-TK as internal control. In each experiment, the total amount of transfected DNA per well was made equal by the addition of empty backbone vectors.

Cell viability assays, such as MTT and cell flow cytometry, cell death analysis (7AAD staining), and caspase 3 activation assays as discussed herein can be used in methods for looking at PSA inhibition activity.

In certain assays and methods, the amount of PSA transcript or translation product can be determined by expression analysis, quantitative PCR, RNA collection, western blot analysis, colony formation assay, GST pull down assay, co-immunoprecipitation, immunoflourescense as appropriate. These and other techniques can be used in the methods disclosed herein.

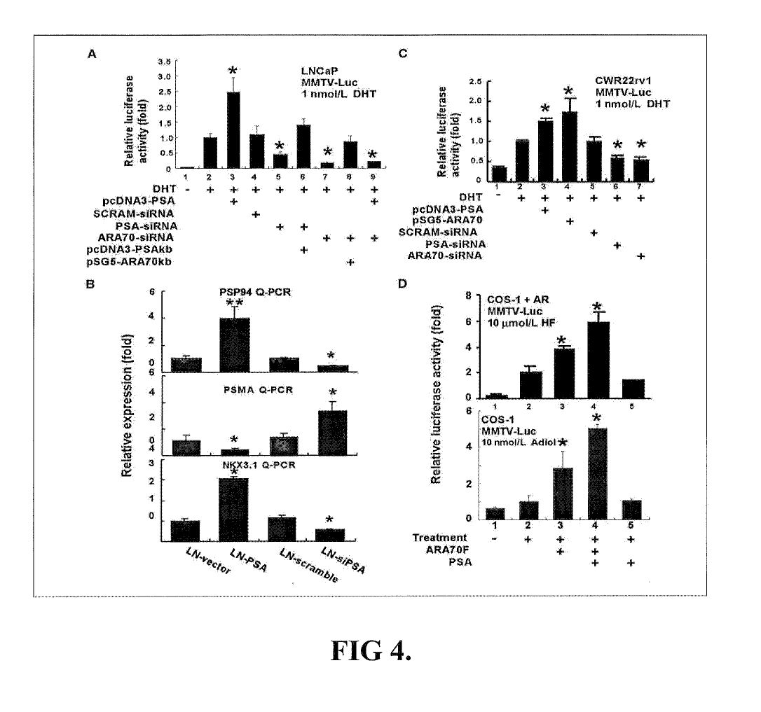

Disclosed herein, PSA enhances the ARA70-induced AR transactivation in the presence of 10 μM HF or 10 nM Adiol (See FIG. 4d). Consistent with this, also disclosed herein, increased tissue PSA in the hormone refractory stage helps tumor cells survive in the castration environment by activating AR transcription.

The data disclosed herein show that the protease activity is not crucial for PSA to stimulate growth and promote AR transactivation.

Also disclosed herein, PSA can utilize the AR/p53 pathway to promote growth. Use of p53 regulated genes to identify molecules that inhibit PSA, where inhibition of PSA with results in higher expression of p21 and lower expression of cdk2, cyclinD1, PCNA, and RFC 1. Apoptosis and G1 arrest are also signals of PSA inhibition.

In addition of a PSA inhibitor in the presence of DHT, such as 1 nM, results in suppression of cell growth. PSA inhibitors inhibit tumor growth in vivo.

### 3. PSA Methods

Disclosed are methods of testing a compound comprising adding the compound to a system, wherein the system comprises PSA, wherein the system comprises ARA70, and assaying the effect of the compound on PSA interaction with ARA70.

Also disclosed are methods further comprising the step of comparing the effect of the compound to a control and/or further comprising selecting a compound that decreases PSA interaction with ARA70.

Disclosed are methods of testing a compound for the ability to modulate PSA activity, comprising adding the compound to a system, wherein the system comprises PSA, wherein the activity of PSA increases cell proliferation, and assaying the effect the compound has on PSA increased cell proliferation.

Also disclosed methods, wherein the compound inhibits PSA-ARA70 interaction, further comprising the step of comparing the effect of the compound to a control, wherein the compound interacts with PSA, wherein the compound interacts with ARA70, wherein the compound interacts with PSA and ARA70, wherein system further comprises AR, wherein the system further comprises ARA70, wherein the system further comprises a cell, wherein the cell is a hormone refractory cell, AR-positive CWR22rv1, LNCaP, wherein the compound is a nucleic acid, wherein the compound is an antibody, wherein the compound is a protein, peptide, amino acid, or derivative or mimetic thereof, wherein the compound is a small molecule, further comprising the step of assaying the cell growth, further comprising the step of comparing the effect of the compound to a control, and/or further comprising selecting a compound wherein the compound decreases PSA activity more than the control.

Disclosed are methods for treating cancer comprising administering a composition to a subject wherein the composition inhibits PSA activity, wherein the activity of PSA increases cell proliferation.

Also disclosed are methods, wherein the composition comprises a PSA inhibitor, wherein the PSA inhibitor does not inhibit the protease activity, wherein the PSA inhibitor decreases an interaction between PSA and ARA70, wherein the PSA inhibitor interacts with PSA, wherein the PSA inhibitor interacts with ARA70, wherein the PSA inhibitor interacts with PSA and ARA70, wherein the PSA inhibitor is a functional nucleic acid, wherein the PSA inhibitor is an antibody, wherein the PSA inhibitor is a protein, peptide, amino acid, or derivative or mimetic thereof, wherein the compound is a small molecule, further comprising the step of determining whether the subject is in need of administration of a PSA inhibitor, further comprising determining whether the subject is in further need of administration of a PSA inhibitor after the PSA inhibitor has been monitored, and/or further comprising selecting a compound wherein the compound decreases PSA activity more than the control.

Disclosed are compounds for inhibiting PSA activity comprising a compound that prevents PSA-ARA70 interaction, and wherein the composition mimics ARA70, compounds for inhibiting PSA activity comprising a compound that prevents PSA-ARA70 interaction, and wherein the composition mimics PSA, compounds for inhibiting PSA activity comprising a compound that prevents PSA-ARA70 interaction.

Also disclosed are compounds and compositions of claims, wherein the compound or composition is an antibody, wherein the antibody is a monoclonal antibody, wherein the antibody is a polyclonal antibody, and/or wherein the composition is a functional nucleic acid, wherein the functional nucleic acid is an aptamer.

Also disclosed are compounds or compositions produced by any of the methods disclosed herein, such as the methods of screening, testing, and identifying.

### 4. KLK2 and KLK2 as a Target

Disclosed herein is data that shows that KLK2, not only is a marker for cancer, such as prostate cancer, but it is also a therapeutic target for the treatment of cancer, such as prostate cancer. Through this interaction with ARA70, KLK2 can promote cancer cell growth, such as prostate cancer cell growth and proliferation.

Disclosed are methods of identifying KLK2 inhibitors which inhibit the cancer proliferation activity of KLK2, such as the activity promoted by the KLK2-ARA70 interaction. Also disclosed are methods of identifying cancers which are promoted by KLK2. In addition, disclosed are methods of treating cancer and compositions that are KLK2 inhibitors that can be used, for example as in methods of treating cancer, such as prostate cancer.

Disclosed herein tissue KLK2 is involved in the development of hormone-refractory prostate cancer. This means, that not only does inhibition of KLK2 reduce cancer, such as prostate cancer proliferation, inhibition of KLK2 also reduces cancer cell, such as prostate cancer proliferation in cells which are no longer responsive in a negative way to, for example, hydroxyl flutamide (HF). Thus, inhibition of KLK2 cancer promoting activity can act to decrease cell proliferation of cancer, such as prostate cancer, which is no longer responsive to traditional ablation therapy. Consistent with this, histological analyses show the increased tissue KLK2 levels are correlated with lower cell apoptosis index and higher cell proliferation rate in hormone-refractory tumors specimens. Also consistent with this, KLK2 was found to promote the growth of AR-positive CWR22rv1 and high passage LNCaP (hormone refractory prostate cancer cells), but not that of AR-negative PC-3.

KLK2 can induce AR transactivation via cooperating with ARA70. This can result in decreased apoptosis and increased cell proliferation in AR positive, yet hormone insensitive prostate cancer cells.

Also disclosed herein, the protease activity of KLK2 is not crucial for KLK2 to stimulate growth and promote AR transactivaton.

One way of inhibiting KLK2 cancer cell proliferation activity is through the knockdown of KLK2 in a cancer cell, such as a prostate cancer cell. In this context, knockdown of KLK2 refers to functional nucleic acid acivities, such as siRNA, RNAi, antisense, or even ribozymes, for example, which will reduce the amount of KLK2 transcript in a cancer cell, such as a prostate cancer cell. If inhibition of KLK2 cancer cell proliferation activity occurs in cancer cells, such as in LNCaP and CWR22rv1 cells this causes cell apoptosis and cell growth arrest at the GI phase. Targeting KLK2 for inhibition results in the suppression of cancer growth, such as prostate cancer growth.

Disclosed are KLK2 inhibitors, such as KLK2 inhibitors that inhibit the cancer cell proliferation activity, such as prostate cancer cell proliferation activity. A KLK2 inhibitor can be any compound that reduces any KLK2 activity as defined herein, such as binding ARA70 or decreasing AR target gene activation or decreasing cell proliferating activity. In certain embodiments the KLK2 inhibitor is a KLK2 inhibitor that does not directly interact with the protease site and/or the protease activity. In certain embodiments, a KLK2 inhibitor is a competitive inhibitor for ARA70 interaction. Thus, disclosed are KLK2 inhibitors which bind KLK2 and competitively compete with ARA70 binding.

A KLK2 inhibitor can also be an antibody, protein, peptide, amino acid, or derivative or mimetic, a functional nucleic acid, such as a siRNA. For example, a functional nucleic acid KLK2 inhibitor can be to the following target sites for KLK2, KLK2-siRNA can be the target sites for KLK2. An ARA inhibitor can be an siRNA that targets the following site in ARA70 and GAGGAGACACTTCAACAGC (384-402).

Also disclosed are ARA70-KLK2 inhibitors, which are molecules that function as competitive inhibitors for KLK2 binding. Thus, disclosed are ARA70-KLK2 inhibitors that bind ARA70 and competitive inhibit KLK2 interaction with ARA70.

Disclosed herein, the accelerated growth of the hormone refractory prostate tumor is correlated with the increased tissue KLK2 expression. KLK2 increases cell growth in AR-positive LNCaP and CWR22rv1 cells, but not in AR-negative PC-3. Disclosed are diagnostic and prognostic assays for a subject comprising assaying KLK2 and correlating the amount to a control or a standard for prediction about the effects of hormone refractory stage prostate cancer.

Also disclosed are results that revealed that the higher expression of KLK2 in human prostate refractory tumors facilitates tumor cell survival even during HF treatment by resistant to cell death and accelerating cell cycle.

Disclose are methods of treatment of prostate cancer wherein the inhibition of KLK2 cancer cell proliferation activity is performed in cancers that are AR positive, for presence and/or activity.

Disclosed herein, KLK2 enhances AR transactivation via interaction with ARA70.

Also disclosed herein, inhibition of KLK2 inhibits AR positive regulated genes, such as PSP94²³ and Nkx3.1²⁴. Assaying these genes for the amount of KLK2 activity, wherein the activity of KLK2 increases AR activity in a cancer cell, such as cancer cell proliferation activity, such as in a prostate cancer cell. Likewise, inhibition of KLK2 increases endogenous AR negative-regulated target genes such as PSMA^{25,26} with the concomitant assays for KLK2 inhibition activity.

In certain assays, high passage, and or stably transfected, LNCaP cells can be used and can be grown in RPMI-1640 (Life Technologies, Rockville, Md.) with 10% CD serum, 100 units/ml of penicillin, and 100 ug/ml streptomycin under 5% CO2.

In certain assays, COS-1, PC-3, LNCaP and CWR22rv1 cells can also be used, and can be grown in appropriate medium at 1-4×10⁵ cells in 24-well plates, were transfected with indicated plasmids using SuperFect (Qiagen) according to the manufacturer's procedure. After incubation for 2-3 hr, the medium was changed and cells were treated with ethanol, DHT, or other ligands for 24 hr. After washing with 1× PBS twice, the cells were harvested in 100 μl of passive cell lysis buffer (Promega) at 4° C. for 20 mins. The luciferase activity in 20 μl cell lysate was measured by the Dual-Luciferase Reporter 1000 Assay system (Luminometer, Tunner Designs) with MMTV-Lue as reporter of AR and pRL-TK as internal control. In each experiment, the total amount of transfected DNA per well was made equal by the addition of empty backbone vectors.

Cell viability assays, such as MTT and cell flow cytometry, cell death analysis (7AAD staining), and caspase 3 activation assays as discussed herein can be used in methods for looking at KLK2 inhibition activity.

In certain assays and methods, the amount of KLK2 transcript or translation product can be determined by expression analysis, quantitative PCR, RNA collection, western blot analysis, colony formation assay, GST pull down assay, co-immunoprecipitation, immunoflourescense as appropriate. These and other techniques can be used in the methods disclosed herein.

Disclosed herein, KLK2 enhances the ARA70-induced AR transactivation in the presence of 10 μM HF or 10 nM Adiol. Consistent with this, also disclosed herein, increased tissue KLK2 in the hormone refractory stage helps tumor cells survive in the castration environment by activating AR transcription.

Also disclosed herein, KLK2 can utilize the AR/p53 pathway to promote growth. Use of p53 regulated genes to identify molecules that inhibit KLK2, where inhibition of KLK2 with results in higher expression of p21 and lower expression of cdk2, cyclinD1, PCNA, and RFC1. Apoptosis and G1 arrest are also signals of KLK2 inhibition.

In addition of a KLK2 inhibitor in the presence of DHT, such as 1 nM, results in suppression of cell growth. KLK2 inhibitors inhibit tumor growth in vivo.

Disclosed herein KLK2 increases cell growth in LNCaP cells but not in PC-3 cells.

KLK2 is highly expressed in AR positive LNCaP cells, mildly expressed in CWR22RV1 and not expressed in AR negative PC-3, DU145, and COS-1 cells.

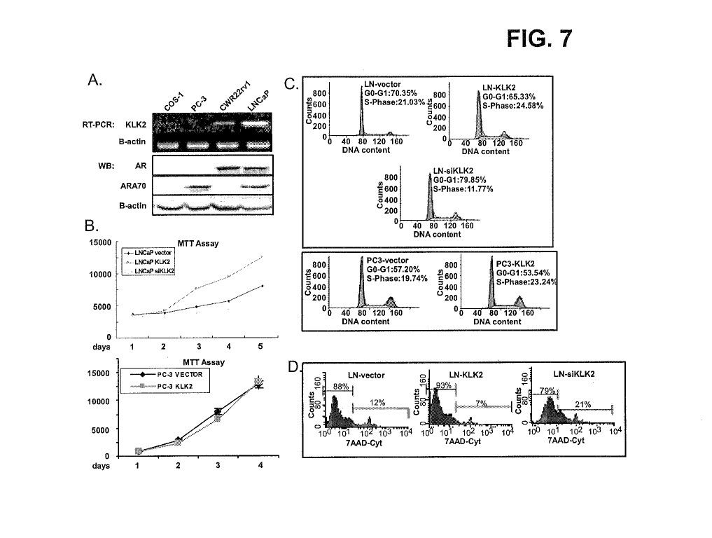

LNCaP and PC-3 cells can be used to test the KLK2 AR effect. MTT assays can also be used to assay the effect in the cells it can be in the presence or absence of DHT such as 10 nM DHT (FIG. 7B). Flow cytometry analysis, for example with propidium iodide staining can also be used for cell assays. Another way to identify compounds which when tested as KLK2 inhibitors is to assay an increased 01 arrest, for example, by at least 5%, 10%, 15%, 20%, 25%, 30%, 40%, 50%, 100%, 200%, or 500%. Another assay which can be used is any assay, for example FACS with 7-AAD staining, to determine apoptosis, and compounds that are KLK2 inhibitors will increase apoptosis, for example in LNCaP cells or similar.

Also disclosed herein, KLK2 enhances ARA70-induced AR transactivation. Assays for AR transactivation can be used to determine the effect of a compound on KLK2 activity, such as KLK2 cell proliferating activity.

KLK2 exerts its cell proliferating activities on AR positive cells, such as LNCaP cells. KLK2 enhances the AR coactivator ARA70-induced AR transactivation in the presence of 10 nM DHT and 1 nM DHT in cell assays, such as assays in green monkey kidney COS-1 cells. KLK2 can also enhance ARA70-induced AR transactivation in the presence of 10 μM HF or 10 nM E2.

KLK2 can also enhance the ARA70 induced AR transactivation in LNCaP cells, as well as in PC-3 cells with stably transfected AR-cDNA. KLK2-siRNA can suppress endogenous KLK2 expression in LNCaP cells, and it was found that AR transactivation was reduced in the presence of 10 DHT and KLK2-siRNA.

To test whether The KLK2 effect occurs through ARA70, as when endogenous ARA70 expression in LNCaP cells was knocked down by ARA70 siRNA, KLK2 lost such enhanced AR transactivation.

Disclosed are KLK2-siRNA stably transfected cells, such as stably transfected LNCaP cells. KLK2-siRNA in LN-siKLK2 cells suppressed the expression of these AR target genes. Thus, assaying AR target genes can be used in the disclosed methods to assess the activity of KLK2 or its inhibition in a cell or animal.

Disclosed herein KLK2 functions as a modulator to enhance ARA70-enhanced AR transactivation. KLK-2 can interact with ARA70 as determined by yeast and mammalian two hybrid assays and GST pull down assays.

Higher expression of KLK2 results in the higher expression of MDM2, lower expression of p53, reduced p21 and bax, and increased cdk2 and bcl2. KLK2 can modulate cell growth and apoptosis through the AR-p53 pathway.

### KLK2 Methods

Disclosed are methods of testing a compound comprising adding the compound to a system, wherein the system comprises KLK2, wherein the system comprises ARA70, and assaying the effect of the compound on KLK2 interaction with ARA70.

Also disclosed are methods comparing the effect of the compound to a control, and/or further comprising selecting a compound that decreases KLK2 interaction with ARA70

Disclosed are methods of testing a compound for the ability to modulate KLK2 activity comprising, adding the compound to a system, wherein the system comprises KLK2, wherein the activity of KLK2 increases cell proliferation, and assaying the effect the compound has on KLK2 increased cell proliferation.

Also disclosed are methods, wherein the compound inhibits KLK2-ARA70 interaction, further comprising the step of comparing the effect of the compound to a control, wherein the compound interacts with KLK2, wherein the compound interacts with ARA70, wherein the compound interacts with KLK2 and ARA70, wherein system further comprises AR, wherein the system further comprises ARA70, wherein the system further comprises a cell, wherein the cell is a hormone refractory cell, AR-positive cell, CWR22rv1 cell, or LNCaP cell, wherein the compound is a nucleic acid, wherein the compound is an antibody, wherein the compound is a protein, peptide, amino acid, or derivative or mimetic thereof, wherein the compound is a small molecule, further comprising the step of assaying the cell growth, further comprising the step of comparing the effect of the compound to a control, further comprising selecting a compound wherein the compound decreases KLK2 activity more than the control.

Disclosed are methods for treating cancer comprising, administering a composition to a subject wherein the composition inhibits KLK2 activity, wherein the activity of KLK2 increases cell proliferation.

Also disclosed are methods, wherein the composition comprises a KLK2 inhibitor, wherein the KLK2 inhibitor does not inhibit the protease activity, wherein the KLK2 inhibitor decreases an interaction between KLK2 and ARA70, wherein the KLK2 inhibitor interacts with KLK2, wherein the KLK2 inhibitor interacts with ARA70, wherein the KLK2 inhibitor interacts with KLK2 and ARA70, wherein the KLK2 inhibitor is a functional nucleic acid, wherein the KLK2 inhibitor is an antibody, wherein the KLK2 inhibitor is a protein, peptide, amino acid, or derivative or mimetic thereof, wherein the compound is a small molecule, further comprising the step of determining whether the subject is in need of administration of a KLK2 inhibitor, further comprising determining whether the subject is in further need of administration of a KLK2 inhibitor after the KLK2 inhibitor has been monitored, further comprising selecting a compound wherein the compound decreases KLK2 activity more than the control.

Disclosed are compounds for inhibiting KLK2 activity comprising a compound that prevents KLK2-ARA70 interaction, and wherein the composition mimics ARA70.

Also disclosed are compounds for inhibiting KLK2 activity comprising a compound that prevents KLK2-ARA70 interaction, and wherein the composition mimics KLK2.

Also disclosed are compounds for inhibiting KLK2 activity comprising a compound that prevents KLK2-ARA70 interaction.

Also disclosed are compounds, wherein the compound is an antibody, wherein the antibody is a monoclonal antibody, wherein the antibody is a polyclonal antibody, wherein the compound is a functional nucleic acid, wherein the functional nucleic acid is an aptamer.

Also disclosed are compounds produced by the method of screening any of the methods disclosed herein.

### 6. Molecules Inhibiting PSA, ARA70, or Other Disclosed Activity

Based on the understanding disclosed herein that PSA, ARA70, or other disclosed activity has activity which, can be androgen independent. Any means for inhibiting PSA, ARA70, or other disclosed activity can be utilized, because as is disclosed herein.

Disclosed are functional nucleic acids that interact with either the mRNA, DNA, or proteins, related to PSA, ARA70, or other disclosed activity, for example. In certain embodiments the functional nucleic acids can mimic the binding of, for example, PSA, ARA70, or other disclosed activity.

### a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the proteins disclosed herein, such as PSA, ARA70, or other disclosed activity or the genomic DNA of any of the proteins disclosed herein, such as PSA, ARA70, or other disclosed activity or they can interact with the polypeptide any of the proteins disclosed herein, such as PSA, ARA70, or other disclosed activity. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAscH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of PSA, ARA70, or other disclosed activity, for example, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424 , 5,780,228, 5,792,613, 5,795,721, 5,846,713, 5,858,660 , 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozym es that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972.704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman. EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### b) Antibodies

Disclosed are antibodies that bind the PSA, ARA70, or other disclosed activity, for example. In certain embodiments, the antibodies bind AR, such that the antibodies mimic the binding of PSA, ARA70, or other disclosed activity. This mimicking can occur through, for example, competitively binding with PSA, ARA70, or other disclosed activity. These antibodies can be isolated by for example, raising antibodies to PSA, ARA70, or other disclosed activity, as disclosed herein, and then assaying the hybridomas for antibodies that are competed off with PSA, ARA70, or other disclosed activity, for example. The antibodies can also be identified by assaying their performance in the disclosed A PSA, ARA70, or other disclosed activity assays herein, and comparing that activity in the presence of the antibody to, for example, the activity in the presence of PSA, ARA70, or other disclosed activity, for example.

### (a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as described herein. The antibodies are tested for their desired activity using the in vitro assays described herein, or by analogous methods, after which their in vivo therapeutic and/or prophylactic activities are tested according to known clinical testing methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V (H)) followed by a number of constant domains. Each light chain has a variable domain at one end (V (L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as scFv, sFv, F(ab′)2, Fab′, Fab and the like, including hybrid fragments. Thus, fragments of the antibodies that retain the ability to bind their specific antigens are provided. For example, fragments of antibodies which maintain PSA, ARA70, or other disclosed activity, for example, binding activity or mimic PSA, ARA70, or other disclosed activity, for example, binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

### (b) Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147 (1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (c) Humanized Antibodies

Optionally, the antibodies are generated in other species and “humanized” for administration in humans. Humanized forms of non-human (e.g., murine) antibodies are chimeric immunoglobulins, immunoglobulin chains or fragments thereof (such as seFv, sFv, Fv, Fab, Fab′, F (ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues arc often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen (s), is achieved. In general, the CDR residues are directly and most substantially involved in influencing antigen binding (see, WO 94/04679, published 3 Mar. 1994).

### (d) Monoclonal Antibodies

The term monoclonal antibody as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

Monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the complexes described herein.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J (H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147 (1):86-95 (1991)).

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103) Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against ARA67, AR, GSK2B, or hRad9, for example. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fe fragment. Pepsin treatment yields a fragment that has two antigen combining sites and is still capable of cross-linking antigen.

### (e) Antibody Fragments

Also disclosed are fragments of antibodies which have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with PSA, ARA70, or other disclosed activity, for example. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof. See Harlow and. Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

### (f) Administration of Antibodies

Antibodies of the invention are preferably administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form. Local or intravenous injection is preferred.

Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp., 365-389. A typical daily dosage of the antibody used alone might range from about 1 μg/kg to up to 100 mg/kg of body weight or more per day, depending on the factors mentioned above.

### (g) Nucleic Acid Approaches for Antibody Delivery

The PSA, ARA70, or other disclosed activity, for example, antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment.

### (2) Peptides

### (a) Protein Variants

As discussed herein there are numerous variants of the PSA, ARA70, or other disclosed activity, for example, proteins that are known and herein contemplated. In addition, to the known functional PSA, ARA70, or other disclosed activity, for example, strain variants there are derivatives of the PSA, ARA70, or other disclosed activity, for example, proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala A |
| allosoleucine | Alle |
| arginine | Arg R |
| asparagine | Asn N |
| aspartic acid | Asp D |
| cysteine | Cys C |
| glutamic acid | Glu E |
| glutamine | Gln Q |
| glycine | Gly G |
| histidine | His H |
| isolelucine | Ilc I |
| leucine | Lcu L |
| lysine | Lys K |
| phenylalanine | Phc F |
| proline | Pro P |
| pyroglutamic | pGlu |
| acidp | |
| serine | Ser S |
| threonine | Thr T |
| tyrosine | Tyr Y |
| tryptophan | Trp W |
| valine | Val V |

TABLE 2
Amino Acid Substitutions
Original Residue Exemplary Conservative Substitutions,
others are known in the art.
Ala; Ser
Arg; Lys; Gln
Asn; Gln; His
Asp; Glu
Cys; Ser
Gln; Asn, Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally dcamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill ct al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—C₂ —CH═CH— (cis and trans), —COCH₂ —, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sri 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences, such as PSA, ARA70, or other disclosed activity, for example, it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

### c) Compositions Identified by Screening with Disclosed Compositions/Combinatorial Chemistry

### (1) Combinatorial Chemistry

The disclosed compositions can be used as targets for any combinatorial technique to identify molecules or macromolecular molecules that interact with the disclosed compositions in a desired way. The nucleic. acids, peptides, and related molecules disclosed herein can be used as targets for the combinatorial approaches. Also disclosed are the compositions that are identified through combinatorial techniques or screening techniques in which the compositions have the sequences disclosed herein, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, PSA, ARA70, or other disclosed activity, for example, are also disclosed. Thus, the products produced using the combinatorial or screening approaches that involve the disclosed compositions, such as, PSA, ARA70, or other disclosed activity, for example, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94 (23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94 (23)12997-302 (1997)).

Another preferred method for combinatorial methods designed to isolate peptides is described in Cohen et al. (Cohen B. A., et al., Proc. Natl. Acad. Sci. USA 95 (24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of PSA, ARA70, or other disclosed activity, for example, is attached to a DNA binding domain of a transcriptional activation protein, such as Gal 4. By performing the Two-hybrid technique on this type of system, molecules that bind the desired portion of PSA, ARA70, or other disclosed activity, for example, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

Screening molecules similar to PSA, ARA70, or other disclosed activity, for example, for inhibition of binding to PSA, ARA70, or other disclosed activity, for example, is a method of isolating desired compounds.

Molecules isolated which bind PSA, ARA70, or other disclosed activity, for example, can either be competitive inhibitors or non-competitive inhibitors of the interaction between PSA, ARA70, or other disclosed activity, for example. In certain embodiments the compositions are competitive inhibitors of the interaction between AR and ARA67, GSKB2, or hRad9, for example.

In another embodiment the inhibitors are non-competitive inhibitors of the interaction between PSA, ARA70, or other disclosed activity, for example. One type of non-competitive inhibitor will cause allosteric rearrangements which mimic the effect of the interaction between Ar and of the interaction between PSA, ARA70, or other disclosed activity, for example.

As used herein combinatorial methods and libraries included traditional screening methods and libraries as well as methods and libraries used in iterative processes.

### (2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, PSA, ARA70, or other disclosed activity, for example, are also disclosed. Thus, the products produced using the molecular modeling approaches that involve the disclosed compositions, such as, of the interaction between PSA, ARA70, or other disclosed activity, for example, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem, Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### C. GENERAL METHODS RELATED TO COMPOSITIONS

There are a variety of actions, such as determining homology/identity of nucleic acids or proteins, hybridization, expression, delivery, and pharmaceutical formulations which are applicable for the general and specific compositions disclosed.

### 1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein,

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### a) Sequences

There are a variety of sequences related to the PSA, ARA70, or other disclosed activity, for example, and other disclosed genes, these sequences and others are herein incorporated by reference in their entireties as well as for individual subsequences contained therein.

It is understood that the description related to this sequence is applicable to any sequence disclosed herein unless specifically indicated otherwise. Those of skill in the art understand how to resolve sequence discrepancies and differences and to adjust the compositions and methods relating to a particular sequence to other related sequences (i.e. sequences of PSA, ARA70, or other disclosed activity). Primers and/or probes can be designed for any PSA, ARA70, or other disclosed activity sequence given the information disclosed herein and known in the art.

### 3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors arc agents that transport the disclosed nucleic acids, such as PSA, ARA70, or other disclosed activity, for example, into the cell without degradation and include a promoter yielding expression of the gene in the cells into which it is delivered. In some embodiments the PSA, ARA70, or other disclosed activity, for example, are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason arc a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which arc involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pot, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruscs has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to n heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed PSA, ARA70, or other disclosed activity, for example, or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Seater, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that arc delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination arc known to those of skill in the art.

### c) In vivo/ex vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdeimally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### D. EXAMPLES

### 1. Example 1

### Tissue Prostate-Specific Antigen Facilitates Refractory Prostate Tumor Development via Modulating Androgen Receptor Transactivation

Despite being well recognized as the best biomarker for prostate cancer, pathophysiological roles of prostate-specific antigen (PSA) remain unclear. Disclosed herein tissue PSA is involved in the development of hormone-refractory prostate cancer. Histological analyses show the increased tissue PSA levels are correlated with lower cell apoptosis index and higher cell proliferation rate in hormone-refractory tumors specimens. By stably transfecting PSA into four androgen independent prostate cancer cell lines, PSA was found to promote the growth of AR-positive CWR22rv1 and high passage LNCaP (hormone refractory prostate cancer cells), but not that of AR-negative PC-3 and DU145 cells. It was shown that PSA can induce AR transactivation via cooperating with ARA70, and can result in the decreased apoptosis and increased cell proliferation in AR positive, yet hormone insensitive prostate cancer cells. Surprisingly, protease activity is not crucial for PSA to stimulate growth and promote AR transactivaton. Knockdown of PSA in LNCaP and CWR22rv1 cells causes cell apoptosis and cell growth arrest at the G1 phase. In vitro colony formation assay and in vivo xenografted tumors via targeting PSA all result in the suppression of prostate cancer growth. Collectively, these findings indicated that PSA-siRNA or smaller molecules that can degrade PSA can be developed as alternative approaches to suppress prostate cancer growth.

### a) Results

### (1) The Accelerated Growth of the Hormone Refractory Prostate Tumor is Correlated with the Increased Tissue PSA Expression

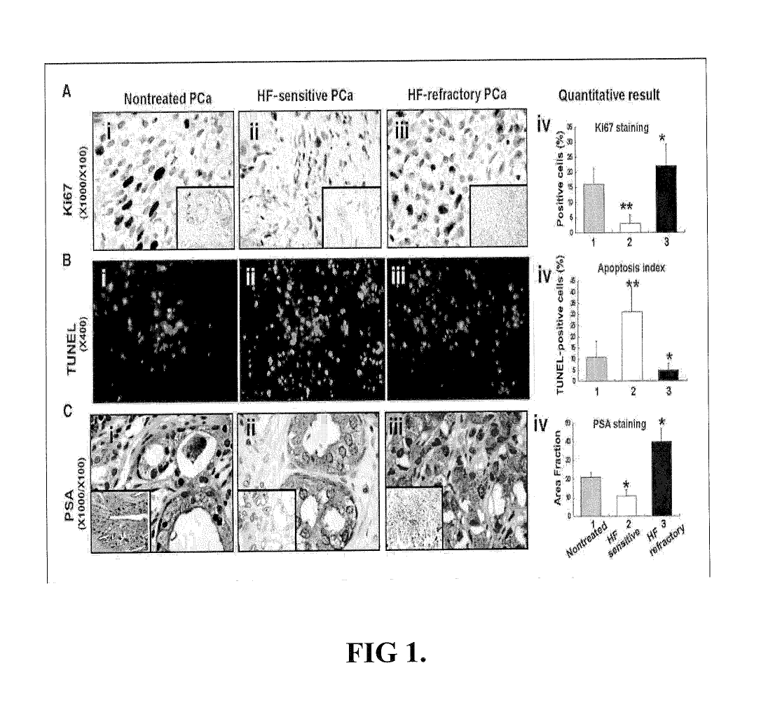

By histologically analyzing the expression of Ki67 in non-hormone treatment, HF treatment sensitive, and HF treatment refractory prostate tumor specimens, it was found that the cell proliferation in the hormone refractory tumors was higher than that in the hormone sensitive tumors (FIG. 1a-d). Also, the cell apoptosis index of the hormone refractory tumors was lower than that of the hormone sensitive tumors using TUNEL assay (FIG. 1c-h). Tissue PSA levels are significantly higher in the hormone refractory tumors as compared to those in hormone sensitive prostate tumors (FIG. 1i-l). Higher PSA levels (FIG. 1I) in the hormone refractory tissues are coincident with higher proliferation rates (FIG. 1d) and lower apoptosis index (FIG. 1h) in these tissues (P<0.05).

### (2) PSA Increases Cell Growth in AR-Positive LNCaP and CWR22rv1 Cells, but not in AR-Negative PC-3 and Du145 Cells

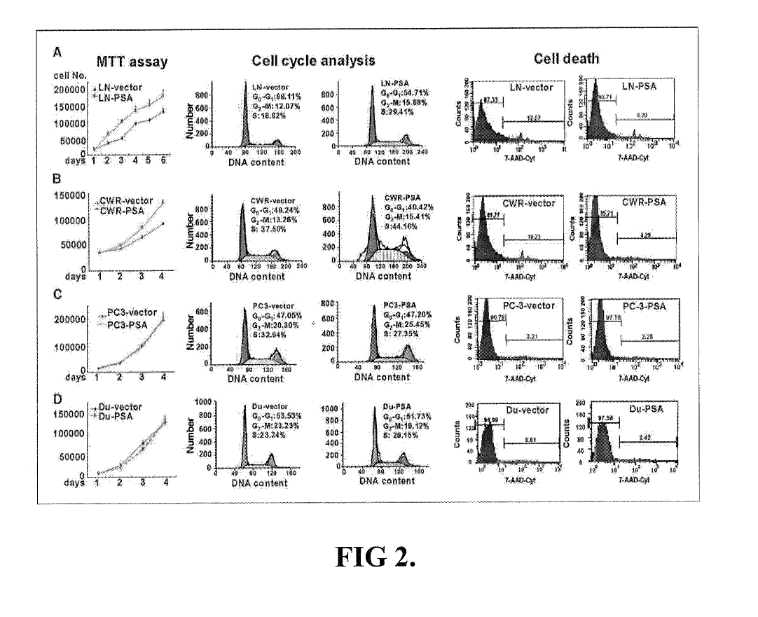

To test the PSA effects on the growth of prostate cancer cells, PSA cDNA was stably transfected into high passage number LNCaP (named LN-PSA), CWR22rv1 (named CWR-PSA), PC-3 (named PC-PSA) and Du145 cells (named Du-PSA). The growth rates of high passage number (n>70) of LNCaP cells and CWR22rv1 cells are not sensitive to the androgen. Therefore, those two cell lines could represent AR-positive hormone refractory prostate cells. The cell viability assays by MTT showed that addition of PSA resulted in an increased number of living cells in AR-positive LNCaP (FIG. 2a) and CWR22rv1 cells (FIG. 2d), but not in AR-negative PC-3 (FIG. 2g) and Du145 (FIG. 2j) cells with 1 nM DHT (human prostate DHT concentration after androgen ablation therapy). The data of cell number differences between controlled groups were further interpreted into the different patterns of cell cycle distribution and cell death by flow cytometry analyses (FIG. 2). Over-expression of PSA in LNCaP and CWR22rv1 cells (LN-PSA and CWR-PSA) resulted in the decreased G0-G1 phase from 69% to 54% (FIG. 2b) and 49% to 40% (FIG. 2e), and increased S phase from 18% to 29% (FIG. 2b) and 37% to 44% (FIG. 2e), respectively. However, ectopic PSA expression in PC3 and Du145 cells (PC3-PSA and Du-PSA in FIGS. 2h and k) had little influence on the cell cycle. Meanwhile, increased PSA expression in LN-PSA and CWR-PSA (FIGS. 2c and f) cells resulted in the decreased cell death, while over-expression of PSA in PC3-PSA (FIG. 2i) and Du-PSA (FIG. 2l) showed no change in the cell death. The different PSA effects between different prostate cancer cell lines, which express AR differently, indicates that PSA's growth stimulation activity could be an AR-dependent event. It has been demonstrated¹⁴ that AR remains in human prostate refractory tumors. The disclosed research was extended to AR-positive hormone refractory cell models, high passage LNCaP and CWR22rv1 cells.

### (3) Knockdown of Endogenous PSA via siRNA Results in the Suppression of Cell Growth

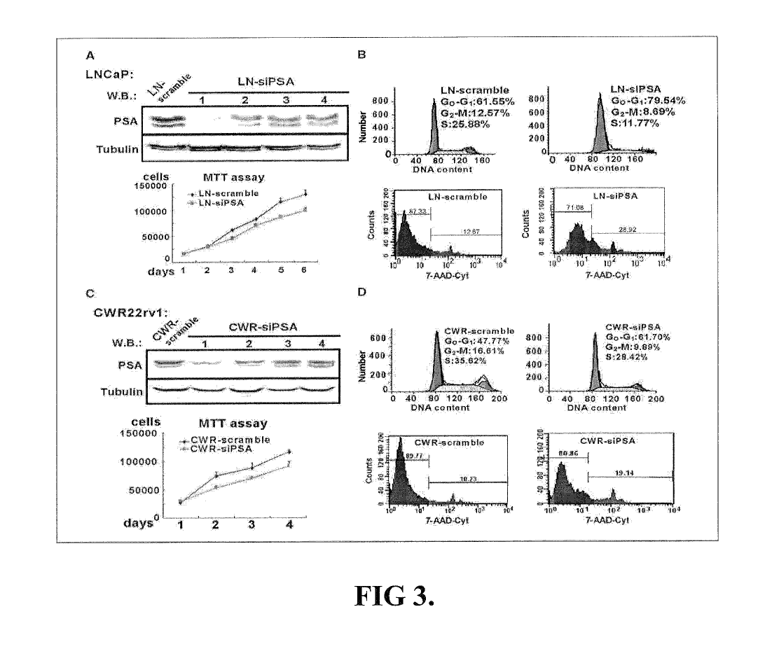

To further confirm the PSA effects on the growth of AR-positive prostate cancer cells, PSA siRNA was stably transfected into high passage LNCaP (named LN-siPSA, FIG. 3a) and CWR22rv1 cells (named CWR-siPSA, FIG. 3e). Knockdown of endogenous PSA in LN-siPSA and CWR-siPSA cells all resulted in cell growth retarded in MTT assays (FIGS. 3b and f, respectively), consistent with an increase in G0-G1 phase from 61% to 79%, and 47% to 61% (FIGS. 3c and g), and increased cell death (FIGS. 3d and h, respectively).

The results revealed that the higher expression of PSA in human prostate refractory tumors may facilitate tumor cell survival from HF treatment by resistant to cell death and accelerating cell cycle. In contrast, it was found PSA did not significantly alter the growth of AR-negative PC-3 and Du145 cell lines. It was found PSA, not like other secretory proteins¹⁸⁻²², could be also located in the cytosol outside of the golgi's apparatus (Suppl. FIGS. 1c and d). Furthermore, using yeast two-hybrid screen (data not shown), it was also found PSA inside the cell might function as an associate protein of ARA70 (Suppl. FIGS. 1a and b).

### (4) PSA Enhances AR Transactivation via Interaction with ARA70

To investigate whether PSA, as an ARA70 associated protein, could go through AR signals to increase cell growth; AR functional study by MMTV-ARE luciferase assay was applied. As shown in FIG. 4, addition of PSA could increase the AR transactivation in LNCaP (FIG. 4a) and CWR22rv1 cells (FIG. 4c), while suppression of endogenous PSA expression in LNCaP and CWR22rv1 cells reduced AR transactivation in the presence of 1 nM DHT (FIGS. 4a and 4c), normalized by both positive (FIG. 4a lane 6) and negative control (FIG. 4a lane 4). To reduce the potential artificial effects due to transient transfection assays, either PSA-cDNA or PSA-siRNA was stably transfected into high passage LNCaP and CWR22rv1 cells. The PSA effects on AR transactivation in multiple sublines was then examined and the results showed PSA could further enhance AR transactivation in both LNCaP and CWR22rv1 cells (data not shown). Using these stably transfected cell lines, it was found PSA could also induce endogenous AR positive-regulated target genes, such as PSP94²³ and Nkx3.1²⁴, as well as suppress endogenous AR negative-regulated target genes such as PSMA^{25,26} (FIG. 4b). In contrast, addition of PSA-siRNA into LNCaP cells results in opposite effects on AR target gene expressions (FIG. 4b).

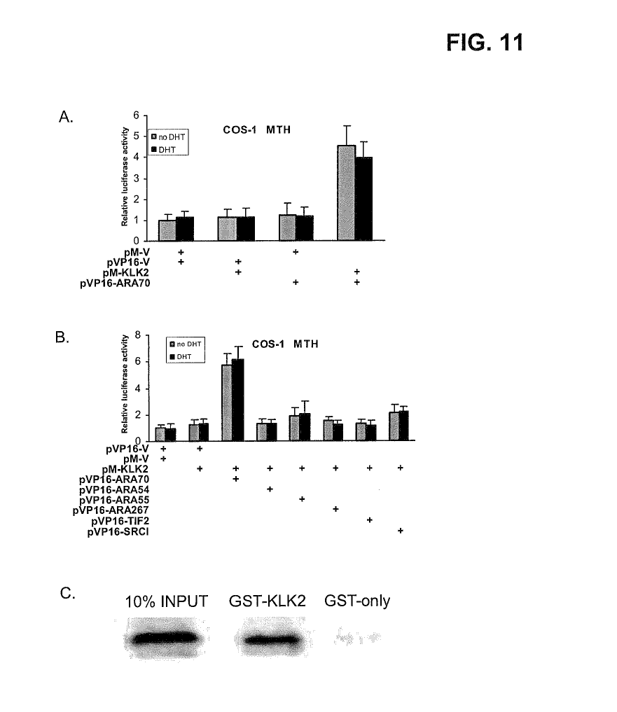

As early studies suggested that the higher expression level of ARA70 could enhance the antiandrogen hydroxyflutamide (HF)- and Deltas-androstenediol (Adiol)-induced AR transactivation^{27,28}, it was therefore tested whether PSA can cooperate with ARA70 to enhance HF- or Adiol-induced AR transactivation. It was found PSA could also enhance the ARA70-induced AR transactivation in the presence of 10 μM IIF or 10 nM Adiol (FIG. 4d). This data revealed that increased tissue PSA in the hormone refractory stage (FIG. 11) could help tumor cells survival in the castration environment by activating AR transcription.

To further strengthen the disclosed results demonstrating that PSA goes through interaction of certain selective AR coregulators, such as ARA70, to induce AR transactivation, it was tested whether reduced endogenous ARA70 (via siRNA) interrupts the PSA-induced AR transactivation. As shown in both LNCaP and CWR22rv1 cells, knockdown of ARA70 by stably-transfected ARA70-siRNA results in the reduction of the PSA-induced AR transactivation (FIG. 4a, lanes 9 vs 3 in LNCaP; FIG. 4c. lanes 7 vs 2 in CWR22rv1), indicating PSA goes through interaction with ARA70 to enhance its coactivity that results in the induction of AR transactivation. These data demonstrated that the existence of ARA70 is critical for PSA enhanced-AR transactivation.

Together, using several cell lines with either transient transfection or stable transfection of PSA, PSA-siRNA, or ARA70-siRNA to assay AR transactivation or AR endogenous target gene expression, it was found PSA could promote cell growth and goes through the ARA70-induced AR transactivation.

### (5) Protease Activity is not Crucial for PSA to Stimulate Growth and Promote AR Transactivation

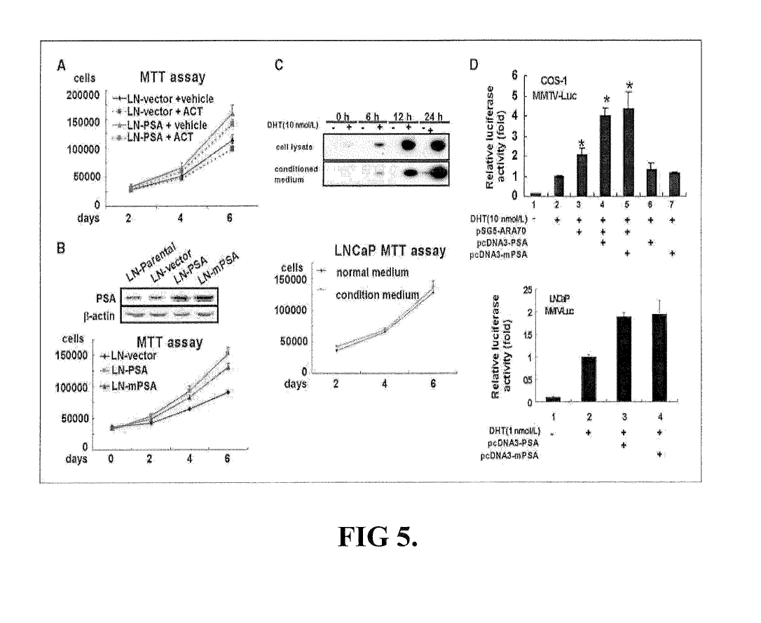

Two different approaches were used to test whether the increased cell growth via increased PSA is protease activity dependent. First PSA protease inhibitor, α₁-antichymotrypsin (1000 ng/ml), was added to the LN-PSA cells and parental control LNCaP cells, and the results showed α₁-antichymotrypsin has limited influence on the PSA-induced cell growth in LN-PSA cells (FIG. 5a). The essential protease domain (213 serine to 213 alanine) was mutated which inactivates the protease activity of PSA²⁹ and stably transfected this mutated mPSA-S213A cDNA into LNCaP cells (LN-PSAm). The results again showed protease activity-null PSA still stimulates the growth of AR-positive prostate cancer cells (FIG. 5b). Furthermore, both cytosol PSA and secreted (into medium) PSA were detected 24 hrs after adding 10 nM DHT to the LNCaP cells with the passage number less than 50 and still respond to the DHT stimulation (FIG. 5c). However, adding these media with secreted PSA into LN-PSA cells results in little influence on the cell growth (FIG. 5d), suggesting the PSA induced cell growth effect might be elicited by the PSA existing inside the LN-PSA cells (named Tissue-PSA). Consistent with cell viability data, ectopic expression of the mutated PSA (mPSA-S213A) also enhances the ARA70-induced-AR transactivation in COS-1 cells (FIG. 5e) and LNCaP cells (FIG. 5f). These results clearly demonstrated that PSA, without its protease activity, could enhance ARA70-induced AR transactivation and stimulate cell growth.

### (6) Mechanisms by Which PSA Enhanced ARA70-Induced AR Transactivation Results in the Increased Prostate Cancer Cell Growth

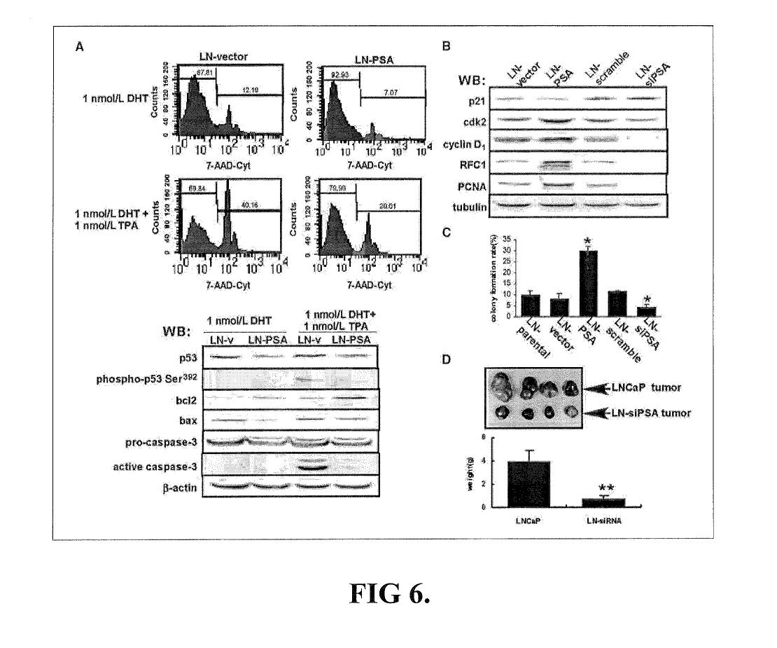

Early studies suggested that androgen/AR might induce cell growth via modulation of p53-mediated cell growth arrest and apoptosis³⁰⁻³². Other studies also demonstrated that AR could via several key factors, such as MDM2, HoxA5, and Egr-1, modulate p53 expression³³. It was tested whether PSA enhanced ARA70-induced AR transactivation resulted in the increased cell growth via modulation of p53-mediated cell growth arrest and apoptosis. The LN-vector control cells and LN-PSA cells were challenged with 1 nM TPA and 1 nM DHT, a condition that was reported previously to accelerate cell apoptosis³⁴. Western blot analysis was also used to examine the apoptosis related markers and showed lower expression of p53 and bax and higher expression of bcl-2 in LN-PSA cells as compared to control LN-vector cells (FIG. 6a). Furthermore, lower expression of p53 was consistent with lower phosphorylation of p53 at Ser392 and lower activated form of caspase-3 (FIG. 6a) in LN-PSA cells treated by TPA as compared to LN-vector cells.

Under environmental changes, such as DNA damage or oxidative stress, p53 can be activated/stabilized to modulate a series of genes that facilitate cell cycle arrest and apoptosis³¹. Functioning as a key downstream target of p53, the p21 might mediate G1 arrest via inhibition of cdks^{31,35}. It was found that LN-PSA cells with decreased G1 phase expressed a lower p21 and higher cdk2, cyclinD1, PCNA, and RFCI, while LN-siPSA cells with G1 arrest expressed a higher p21 and lower expression of cdk2, cyclinD1, PCNA, and RFC1 as compared to parental LNCaP cells (FIG. 6b). Together, these results demonstrate that PSA might go through the AR-p53 pathway to promote cell growth via G1/S cell cycle checkpoint.

### (7) PSA as a New Therapeutic Target to Control Prostate Cancer Growth

All the data disclosed herein indicate that PSA can induce cell growth via ARA70/AR→p53→cell apoptosis and G1 arrest, which suggests that PSA is a new therapeutic target to treat prostate cancer. To test this hypothesis, different approaches were applied to see if reducing endogenous PSA expression can result in the suppression of prostate cancer growth. Using the MTT cell viability assay, it was found that stably transfecting PSA-siRNA into high passage LNCaP cells (LN-siRNA) and CWR22rv1 cells (CWR-siRNA) results in suppression of cell growth in the presence of 1 nM DHT (FIG. 3). Using colony formation assays more colonies in LN-PSA cells were found, and less colonies in LN-siPSA cells were found, as compared to control LNCaP cells (FIG. 6c). Finally, in vivo tumor growth assays using xenografted LNCaP (into the left flank) and LN-siPSA (into the right flank) cells in castrated nude mice also showed smaller tumors in LN-siPSA xenografts as compared to LNCaP xenografts (FIG. 6d). Together, cell line MTT assays, colony formation assays and in vivo xenograft tumor growth assays all demonstrate that targeting PSA via PSA-siRNA to reduce endogenous PSA expression is a potential new therapeutic approach to suppress prostate cancer cell growth.

### b) Discussion

### (1) Pathophysiological Roles of PSA in Prostate Cancer

Early studies suggested that PSA might modulate growth of PSA-producing cells and their surrounding cells^{36,37} via its serine protease activity. PSA might promote the growth and invasion of prostate cancer via degradation of IGFBP-3, fibronectin, and laminin^{2,10,38}. Interestingly, Fortier et al presented evidence that PSA protein itself without its protease activity could also function as an endothelial cell-specific inhibitor of angiogenesis³⁹. Their findings, however, were countered by later findings from Isaacs et al showing the antiangiogenic effects of PSA are not significant enough in PSA-producing cells to appreciably effect tumor growth in vivo¹¹. Therefore, the significance of pathophysiological roles of PSA, if any at all, in prostate cancer, from the above studies, might depend predominantly, if not completely, on the protease activity from PSA. The development of inhibitors to block the protease activity of PSA might then have potential therapeutic advantages to battle the prostate cancer. The findings disclosed herein show PSA, without involving its protease activity, can promote prostate cancer cell growth. Therefore, PSA can have two functions, one in invasion and one in proliferation, and that its protease activity could be important for one and not the other. The data disclosed herein indicate targeting PSA itself, instead of just blocking its protease activity, is needed to stop the PSA-induced prostate cancer progression.

### (2) Clinical Linkage: Tissue-PSA Increased in Prostate Cancer Patients Treated with Androgen Ablation Therapy

An early study showed that 31 of 63 (49%) of the patients died of prostate cancer with their Tissue-PSA values increased (from 0.054 to 0.204 μg Tissue-PSA/μg DNA) during androgen ablation treatment that includes either surgical or chemical castration⁴⁰. The average of their pre-treatment Tissue-PSA values were significantly lower as compared to other groups of patients (0.063 vs. 0.381 μg Tissue-PSA/μg DNA,) who were alive at end of observation period or died of causes other than prostate cancer⁴⁰. This is in agreement with our II-IC staining (FIG. 1) showing the tissue PSA level was also higher in the hormone refractory samples than the hormone sensitive control group. Based on the results herein, the rationale for those patients with increased tissue-PSA during treatment, failed to respond to androgen ablation therapy, and died of prostate cancer could be that the Tissue-PSA synthesis in these patients became androgen insensitive⁴⁰ and other inducers, such as antiandrogens or Adiol, can then stimulate tissue-PSA synthesis^{27,18}.

### (3) New Signaling Pathways from PSA4HF/Adiol-ARA70/AR4p534Cell Apoptosis and Cell Growth Arrest

One possible explanation for the clinical observations and the disclosed experiments is that with expression of AR and ARA70 in those patients at the hormone refractory stage^{14,14-42,} antiandrogen HF or Adiol can induce AR transactivation (FIG. 4d), which results in the increased Tissue-PSA. The increased Tissue-PSA can then go through a feed-back-positive-regulation to further enhance ARA70-induced AR transactivation that results in the suppression of p53 expression via modulation of MDM2/HoxA5/Egr-I signaling pathways³³. The consequence of AR suppression of p53 can then result in the cell survival via the decrease of cell apoptosis via bax/bcl-2/caspase 3 signaling pathways, as well as in the decrease of cell G1 arrest via modulation of p21/cdk2/CyclinD1 signaling pathways (FIG. 6). Previous reports showed that increase in expression of the p53 and p21/WAF1 proteins is an early event during standard androgen withdrawal therapy⁴³. p53 was identified as a critical molecule in response to androgen deprivation in prostate from a mouse model and LNCaP cells³³. In vitro studies on both LNCaP and LAPC4 cells indicated that AR promotes cell growth by abrogation of p53 mediated apoptosis³². Mutant p53 can facilitate the androgen-independent growth of LNCaP cells⁴⁴. On the other hand, inhibition of p53 function diminishes androgen receptor-mediated signaling in prostate cancer cell lines⁴⁵. Also, a functional role for the wild type p53 gene in suppressing prostatic tumorigenesis was well documented^{46,47}. It was also documented that amplification of the AR gene was associated with p53 mutation in hormone refractory prostate cancer⁴⁸. Above all, the androgen receptor regulated p53 signaling pathway was significant for tumor progression, and PSA may play an important role in this pathway.

In summary, results from these studies showing tissue PSA, without involving its protease activity, can promote ARA70-AR mediated cell growth and facilitate refractory tumor development. This observation indicates that PSA can be treated as a therapeutic target to battle prostate cancer.

### c) Materials and Methods

### (1) Plasmids

pSG5 (Stratagene), pcDNA3 (Invitrogen), pSUPERIOR.retro.puro (Oligocnginc), pGEM-T Easy (Promega) vectors were used to construct the plasmids. pSG5-AR, pSG5-ARA70N (N terminal), pSG5-ARA70f (full length), pGEX-GST ARA70, mouse mammary tumor virus promoter-uciferase (MMTV luc), pVP16-ARA70, and pGL-TK were constructed as described previously^{49,50}. Forward primer 5′-GCGGATCCGGGGAGCCCCAAGCTTACC-3′ and reverse primer 5′-CGTCTAGAGGGTGCTCAGGGGTTGGC-3′ were used to PCR amplify full-length PSA cDNA from Marathon-Ready human prostate cDNA library using Pfu Tag polymerase. The PCR product was gel-purified and cloned into BamHI- and XbaI-igested pcDNA3 vector (modified). The mutant PSA (S213A) plasmid was generated from parental pCDNA3-PSA plasmid by mutating the 213^{th} amino acid residue from serine to alanine using QuikChange XL Site-Directed Mutagenesis Kit (Stratagene). The siRNA target sites for PSA and ARA70 GTGGATCAAGGACACCATC (753-771) and GAGGAGACACTTCAACAGC (384-402) respectively, were selected by Oligocngine siRNA designing software and BLAST search to eliminate any sequence with significant homology to other genes. The negative controls for each of them were generated and selected as Scramble-siRNA. The positive control were applied following “knock-back after knock down” strategy by generate pCDNA3-PSAkb plasmid and pSG5-ARA70kb plasmid respectively. pCDNA3-PSAkb was generated from parental pCDNA3-PSA plasmid by mutating PSA-siRNA target sequence to GTGGATCAAAAACACCATC (753-771) using QuikChange XL Site-Directed Mutagenesis Kit (Stratagene), while pSG5-ARA70kb was generated from parental pSG5-ARA70 plasmid by mutating ARA70-siRNA target sequence to GAGGAGACACCCCAACAGC (384-402). All the constructs were verified by DNA sequencing.

### (2) Cell Cultures, Transient DNA Transfection and Promoter Reporter Assay

All cell lines were obtained from the American Type Culture Collection. The COS-1 and PC-3 cells were maintained at 37° C. in DMEM (Gibco-BRL) supplemented with 10% charcoal deprived (CD) serum, 100 units/ml of penicillin, and 100 ug/ml streptomycin under 5% C02. High passage LNCaP cells were grown in RPMI-1640 (Life Technologies, Rockville, Md.) with 10% CD serum, 100 units/ml of penicillin, and 100 ug/ml streptomycin under 5% CO2. 5a-Dihydrotestosterone (DHT) and delta5-androstenediol (Adiol) were obtained from Sigma and hydroxyflutamide (HF) was from Schering.

COS-1, PC-3, LNCaP and CWR22rv1 cells, grown in appropriate medium at 1-4×10⁵ cells in 24-well plates, were transfected with indicated plasmids using SuperFect (Qiagen) according to the manufacturer's procedure. After incubation for 2-3 hr, the medium was changed and cells were treated with ethanol, DHT, or other ligands for 24 hr. After washing with 1×PBS twice, the cells were harvested in 100 p1 of passive cell lysis buffer (Promega) at 4° C. for 20 mins. The luciferase activity in 20 μl cell lysate was measured by the Dual-Luciferase Reporter 1000 Assay system (Luminometer, Tunner Designs) with MMTV-Luc as reporter of AR and pRL-TK as internal control. In each experiment, the total amount of transfected DNA per well was made equal by the addition of empty backbone vectors.

### (3) Immunohistochemical Staining (IHC)

6 hormone refractory prostate cancer specimens, defined by fail in HF treatment and acquired by TURP management of urethral obstruction, were carefully selected and accompanied by 20 no hormone treatment and 15 HF treatment sensitive prostate cancer specimens. All samples were collected from the Department of Pathology of the Tianjin Institute of Urological Surgery and the Sir Run Run Shaw Hospital of Zhejiang University Medical School. Clinical data were collected from medical records.

Samples were fixed in 5% neutral buffered formalin and embedded in paraffin. The AR, ARA70 and PSA protein expression level were studied by IHC method in all 4 pairs of samples. The rabbit anti-PSA polyclonal antibody (DAKO, A0562), the mouse anti-ARA70 antibody, and the rabbit anti-AR (N20) antibody (Santa Cruz Biotechnology) were used in IHC staining. The bound primary antibody was recognized by the biotinylated secondary antibody (Vector), and visualized by VECTASTAIN ABC peroxidase system (Vector) and peroxidase substrate DAB kit (Vector). The positive staining signals were semi-quantitated by Image J software.

### (4) Establishment of Stably Transfected Cell Lines

High passage LNCaP, CWR22rv1, PC-3 and Du145 cells were cultured to the mid- or late-logarithmic phase of growth. After trypsinization, the cells were resuspended and washed twice in 2.5% FBS medium without antibiotics. 400 ul of the cell suspension (10⁷ cells) was ten transferred into the electroporation cuvettes (VWR), set the voltages of the electroporator (Bio-RAD GENE PULSER II) to 300V and hinge capacity to 950 uF, added 20 ug of total plasmid DNA to each cuvette, and incubated for 5 min at RT. After pulse charge, the cells were incubated on ice for 5 min and transferred to a 35-mm culture dish. After culturing in complete medium at 37° C. with 5% C02 for 72 h, the transfected cells were trypsinized and replated in the appropriate selection medium. For pCDNA3-PSA transfected cells, 1000 ug/ml neomycin was used for selection, while for pSuperior-siPSA or pSuperior-siARA70 transfected cells, 5 ug/m1 of puromycin was used. The selection medium was changed every 2-4 days for 2-3 weeks until colonies of resistant cells formed.

### (5) Cell Viability Assay by MTT and Cell Cycle Flow Cytometry

Cells of stable transfected LNCaP, CWR22rv1, PC-3 and Du145 sublines were seeded in 24-well tissue culture plates at a density of 5000 cells/well in media containing 10% CD-FBS with 1 nM DHT. At the indicated time points, the medium was replenished and determined cell proliferation by MTT [3-(4,5-dimethylthiazol-2-yl)-2,5-diphenyltetrazolium bromide assay. Serum-free medium containing MTT (0.5 mg/ml, Sigma) was added into each well and after 4 h incubation at 37° C., cellular formazan product was dissolved with acidic isopropanol, and the absorbance at OD595 was measured by spectrophotometry (Beckman Du640B). 6 replicate wells were used for each sample at each time point. a-Antichymotrypsin (MP Biomedicals, Inc) at a concentration of 1000 ng/ml was used in the MTT assay as a PSA proteinase inhibitor to treat the LNCaP cell sublines. LN-PSA, LN-siPSA cells were grown in 100-mm dishes. The cells were digested by trypsin-EDTA, harvested as many as 1×10⁶ cells, and fixed them in 70% ethanol at 4° C. After 12 h, cells were centrifuged (1000 g, 7 min, 4° C.), and resuspended in PBS containing 0.05 mg/ml RNase A (Sigma), and then incubated at room temperature for 30min. After washing, the cells were stained with 10 mg/ml propidium iodide, filtered through a 60 mm mesh, and analyzed 10,000 cells by flow cytometry (FACSCalibur, BD Company) with MODFIT software (Verity Software House, Inc).

### (6) Cell Death Analysis (7-AAD Staining) and Caspase-3 Activation

7-AAD (Sigma) was dissolved in acetone, diluted in phosphate-buffered saline (PBS) at a concentration of 200 ug/ml, and kept at 20° C. without light until use. 100 ul of 7-AAD solution was added to 2.5×10⁶ target cells in 1 ml of PBS and incubated for 20 min at 4° C. in dark. Cells were then washed with Wash buffer and total DNA was stained with 7-amino-actinomycin D (7-AAD; 20 uL per sample), followed by flow cytometric analysis using FACSort (Becton Dickinson). To assess apoptosis, 5×10⁵ cells were resuspended in PBS, analyzed by flow cytometry, and total DNA content (7-AAD) was determined using Cell Quest (Becton Dickinson) and FCS Express software (De Novo Software).

LN-vehicle and LN-PSA cells were challenged with I nM TPA and 1 nM DHT for 24 hr to induce apoptosis, and cell apoptosis was detected by 7AAD flow cytometry. Simultaneously, we measured caspase-3 activity by Western blot detection of its activated subunits, 17 kDa and 12 kDa (Rabbit anti-Active Caspase-3, BD Biosciences).

### (7) RNA Extraction, Reverse Transcription, and Real Time Quantitative PCR

Confluently grown cells on 100 mm dish were washed with 1× PBS twice, and harvested them in Trizol (Invitrogen). Total RNA were extracted using Trizol and 5 ug total RNA was reverse transcripted into 20 ul cDNA immediately by the SuperScript III kit (Invitrogen) with oligo-dT primer. Real-time quantitative PCR was performed on iCycler IQ multicolor real-time PCR detection system with 1/5 ul cDNA amplified by SYBR Green PCR Master Mix. We designed primers by Beacon Designer 2 software as follows: NKX3.1 (forward: 5′-ATGGTTCCAGAACAGACGCTAT-3′, reverse: 5′-TGCCCACGCAGTACAGGTAT-3′); PSP94 (forward: 5′-TCCTGGGCAGCGTTGTGA-3′, reverse: 5′-TTGGGTGTTTGTTTCCTTTGAG-3′); PSMA (forward: 5′-AAGGAAGGGTGGAGACCTAG-3; reverse: 5′-ACTGAACTCTGGGGAAGGAC-3′); B-Actin (forward: 5′-TGTGCCCATCTACGAGGGGTATGC-3′, and reverse: 5′-GGTACATGGTGGTGCCGCCAGACA-3′) was used as internal control. We calculated threshold (CT) values by subtracting the control CT value from the corresponding B-Actin CT from each time point. We confirmed the absence of nonspecific amplification products by agarose-gel electrophoresis.

### (8) Western Blot Analysis

Cell lysates were prepared in RIPA buffer. Protein samples were then separated on SDS-10% PAGE gel and transferred them to a polyvinylidene difluoride membrane. After blocking by 5% non-fat milk and 5% FBS in PBST buffer, the membrane was immunoblotted with the primary antibody, followed by incubation with AP-conjugated second antibody (Santa Cruz). The membrane was developed by the AP color developing reagents (Bio-RAD). The laboratory generated the monoclonal anti-ARA70 antibody. The rabbit polyclonal anti-AR (N20) and mouse monoclonal anti-PSA antibodies were purchased from Santa Cruz. Anti-p53 monoclonal and anti-bcl2 monoclonal antibodies were purchased from DAKO, Denmark. Rabbit anti-cdk2, rabbit anti-cyclinD1, rabbit anti-p21, rabbit anti-PCNA, rabbit anti-RFC1, rabbit anti-bax, mouse anti-tubulin, and goat anti-actin were obtained from Santa Cruz. Rabbit anti-phospho-p53 (Ser392) antibodies were bought from Cell Signaling Technology, Inc. PSA protein amounts were detected in equal amounts of proteins from cell lysates and equal volumes of concentrated cultured media by Western blot.

### (9) Colony Formation Assay

We determined the cell survival of LN-PSA and LN-siPSA in a colonogenic assay. Briefly, we plated cells (200 cells/well) in 6-well plates and cultured them in normal medium at 37° C. with 5% C02 for 2 weeks. Then we fixed and stained the cells with 10% formalin (35% v/v) and 0.25% crystal violet in 80% methanol for 30 min, washed them with water, and counted the number of colonies, which contain more than 50 cells. We determined the plating efficiency as the fraction of cells that were attached to the support and grew into colonies larger than 1 mm diameter.

### (10) Tumorigenesis in the Nude Mice

10 athymic nuce mice were pre-castrated at 12 weeks old. One week following castration, 5×10⁷ high passage LNCaP and LN-siPSA cells were injected into the left and right dorsal part of these nude mice, respectively. 12 weeks following the injection, the nude mice developed obvious tumors. The xenografts were harvested and the protein levels of PSA were determined by Western blot assay.

### (11) Glutathione S-Transferase (GST) Pull-Down Assay

The full-length PSA was in vitro translated in the presence of [³⁵S]-methionine using T7 polymerase and the coupled transcription/translation kit (Promega). The PGEX-GST-ARA70 plasmid was transformed into BL21-CodonPlus Competent cells (Stratagcne) to express GST fusion ARA70 protein, harvested, and purified the GST ARA70 fusion protein with glutathione-beads. The radiolabeled PSA protein was incubated for 2 hr with the GST-ARA70 fusion protein attached to glutathione beads in interaction buffer (50 mM HEPES, 100 mM NaCl, 20 mM Tris-Cl/pH 8.0, 0.1% Tween20, 10% glycerol, 1 mM DTT, 0.5 mM PMSF, 1 mM NaF, and 0.4 mM sodium vanadate). The beads were washed with NENT buffer 4 times, resuspended in SDS-PAGE loading buffer, and resolved on 12% SDS-PAGE gel electrophoresis followed by autoradiography.

### (12) Co-Immunoprecipitation

500 ug protein from LNCaP cell lysates was incubated with 2 ug anti-ARA70 monoclonal antibody or normal mouse IgG for 4 h at 4° C. with agitation. Protein A/G plus agarose (50 ul) was added to each sample, and incubated for 1 h. After washing three times with RIPA buffer, the complex was resolved on a 10% SDS-polyacrylamide gel. The separated proteins were transferred to a polyvinylidene difluoride membrane, blotted with anti-ARA70, anti-AR, and anti-PSA monoclonal antibody, and developed the bands by an alkaline phosphatase detection kit (Bio-RAD).

### (13) Immunofluorescence Staining and Confocal Laser Scanning Microscopy

High passage LNCaP cells were cultured on chamberslides and fixed in 4% formaldehyde for 30 mM at 4° C., then incubated in 50 mM NH4CI for 3 hours to quench autofluorescence. Following blocked in 10% horse Serum+1% Triton-X 100 PBS for 2 hr at RT, sections were incubated overnight at 4° C. with primary antibodies, goat anti-PSA polyclonal antibody (Santa Cruz), mouse anti-ARA70 monoclonal antibody and rabbit anti-AR polyclonal antibody (Santa Cruz) or rabbit anti-Giantin (Golgi marker from Abeam). Following 60 min rinse (3×20 min, PBS+1% Triton-X 100), slices were incubated with secondary antibodies (Alexa Fluors, donkey anti-goat 488 and horse anti-mouse TEXAS-Red and chicken anti-rabbit 647) for 1 hr at RT. Slices were rinsed for 60 min (3×20 min), and mounted with Vectashield Mounting Medium H1000 (Vector Laboratories, Burlingame, Calif.) and examined on a confocal microscope (Leica TCS SP).

### 2. Example 2

### Human Kallikrein 2 (KLK2) Promotes Prostate Cancer Cell Growth via Function as a Modulator to Promote the ARA70-Enhanced Androgen Receptor Trans activation

Recent data implied that tissue KLK2 might be involved in carcinogenesis and tumor metastasis in the prostate cancer. The detailed mechanisms, however, remain unclear. Using a yeast two-hybrid screen, KLK2 was found to be one of the androgen receptor (AR) coregulator ARA70 associated proteins. These findings were confirmed with mammalian two-hybrid and GST pull-down assays. AR transactivation assays in COS-1 and LNCaP cell indicated KLK2 could enhance ARA70-induced AR transactivation, which then results in the suppression of p53 expression via modulation of Mdm2/HoxA5/Egr-1 pathways. The consequence of p53 suppression can be decreased apoptosis and cell growth arrested at the G1 phase via modulation of the bax/bcl-2/caspase3 and p21/cdks signal pathways. Together, these results indicate KLK2 is a new therapeutic target for prostate cancer. KLK2-siRNA or smaller molecules that can degrade KLK2 or interrupt the interaction between KLK2 and androgen/AR-ARA70 complex can be developed as alternative approaches to suppress prostate cancer growth.

### a) Introduction

Human kallikrein 2 (KLK2), a member of the kallikrein gene family, is a serine protease with trypsin-like activity that is expressed mainly in the prostate (C A, M I P, et al., Mol Cancer Res 2004; 2:257-280). KLK2 is often coexpressed and colocalized with kallikrein 3, known as prostate specific antigen (PSA), within the same tissues (LoEvgren L, et al., J Androl 1999; 20: 348-355). KLK2 is present at about 1% of the concentration of PSA in seminal plasma (LoEvgren L, et al., J Androl 1999; 20: 348-355). In vitro studies have shown that KLK2 is able to activate itself (Kumar A, et al., Cancer Res 1997; 57:3111-3114). Once enzymatically active, KLK2 and PSA contribute to seminal clot liquefaction after ejaculation, which is integral to sperm motility, through their hydrolysis of seminal vesicle proteins, seminogelin I and II, and fibronectin. The expression of KLK2 is dependent on androgen(s) and androgen receptor (AR) (Murtha P, et al., Biochemistry 1993; 32:6459-6464).

The tissue kallikrein family has been proven to be a rich source of cancer biomarkers (C A, M I P, et al., Mol Cancer Res 2004; 2:257-280). Like human PSA, serum KLK2 may function as an alternate or complementary biomarker for prostatic diseases. Recent data also suggested that tissue KLK2 may be involved in carcinogenesis, tumor metastasis, and invasion of prostate cancer via its protease activity (Diamandis E P, et al., Trend Endocrinol Metab 2000; 11:54-60). Early data suggested that KLK2 might also be involved in the activation of different protease pathways. KLK2 and KLK4 could activate the pro-form of uPA, a serine protease that converts the serine protease, plasminogen, to plasmin, which in turn degrades the ECM and activates members of the matrix metalloprotease family (Solovyeva N I, et al., Immunopharmacology 1996; 32:131-134). In addition to plasmin, KLK2 may also activate pro-matrix metalloproteases via cleavage of their pro-peptides (C A, M I P, et al., Mol Cancer Res 2004; 2:257-280). These findings implicate KLK2 in the promotion of tumor progression, invasion, and metastasis. However, the pathophysiological roles of KLK2 remain unclear. Disclosed herein, KLK2 can function as a modulator to stimulate ARA70-induced AR transactivation which can result in increased p53-mediated cell growth.

### b) Materials and Methods

### (1) Materials

5α-Dihydrotestosterone (DHT), 17β-estradiol (E2) and hydroxyflutamide (HF) were obtained from Sigma and Schering. pSG5-AR, pSG5-ARA70N (N terminal), pSG5-ARA70F (full length), pGEX-GST-ARA70, mouse mammary tumor virus promoter-luciferase (MMTV-luc), pVP16-ARA70, and pGL-TK were constructed as described previously (Yeh et al., 1996; Heinlein et al., 1999; Lin et al., 2003). Full-length KLK2 cDNA was amplified from Marathon-Ready human prostate cDNA library using Pfu Taq polymerase and cloned into pcDNA3 vector (modified). DNA plasmids for KLK2 and ARA70 siRNA were constructed following the instruction of Oligoengine siRNA designing and BLAST search to eliminate any sequence with significant homology to other genes. All the constructs were verified by DNA sequencing.

### (2) Cell Cultures, Transient DNA Transfection, and Promoter Reporter Assay

All cell lines were obtained from the American Type Culture Collection. The COS-1 and PC-3 cells were maintained at 37° C. in DMEM (Gibco-BRL) supplemented with 10% charcoal-dextran treated FCS, 100 units/ml of penicillin, and 100 μg/ml streptomycin under 5% CO₂. High passage LNCaP cells were grown in RPMI-1640 (Life Technologies, Rockville, Md.) with 10% FCS, 100 units/ml of penicillin, and 100 μg/ml streptomycin under 5% CO₂.

Cells, grown in appropriate medium at 1-4×10⁵ cells in 24-well plates, were transiently transfected using SuperFect (Qiagen) according to the manufacturer's procedure. After incubation for 2-3 hr, the medium was changed and cells were treated with ethanol, DHT, or other ligands for 24 hr. After washing with 1×PBS twice, the cells were harvested in 100 μl of passive cell lysis buffer (Promega) at 4° C. for 20 mins. The luciferase activity in 20 μl cell lysate was measured by the Dual-luciferase reporter 1000 assay system (Luminometer, Tunner Designs) with MMTV-luc as reporter of AR and pRL-TK as internal control. In each experiment, the total amount of transfected DNA per well was made equal by the addition of empty backbone vectors.

### (3) Establishment of Stable Transfected Cell Lines

LNCaP, and PC-3 cells were cultured to the mid- or late-logarithmic phase of growth. After trypsinization, the cells were resuspended and washed twice in 2.5% FBS medium without antibiotics. Then 400 μl of the cell suspension (10⁷ cells) was transferred into the electroporation cuvettes (VWR), 20 μg of total plasmid DNA was added to each cuvette, and each was incubated for 5 min at RT. The voltages of the electroporator (Bio-RAD GENE PULSER II) were set to 300V and hinge capacity to 950 μF. After pulse charge, the cells were incubated on ice for 5 min and transferred to a 35-mm culture dish. After culturing in complete medium at 37° C. with 5% CO2 for 72 hr, the transfected cells were trypsinized and replated in the appropriate selection medium. For pcDNA3-KLK2 transfected cells, 1000 μg/ml neomycin was used for selection, while for pSuperior-siKLK2 or pSuperior-siARA70 transfected cells, 5 μg/ml of puromycin was used. The selection medium was changed every 2-4 days for 2-3 weeks until colonies of resistant cells formed. By this method, LNCaP-pcDNA3-KLK2 (LN-KLK2), LNCaP-pcDNA3-vector (LN-vehicle), LNCaP-pSUPERIOR-scramble (LN-scram), LNCaP-pSUPERIOR-siKLK2 (LN-siKLK2), LNCaP-pSUPERIOR-siARA70 (LN-siARA70) cell lines were established from low passage LNCaP parental cell lines. The PC-3-pcDNA3-KLK2 (PC-KLK2) and PC-3-pcDNA3-vector (PC-vehicle) cell lines were also established.

### (4) Cell Growth Assay and Cell Cycle Flow Cytometry

Cells of stable transfected LNCaP and PC-3 sublines were seeded in 24-well tissue culture plates at a density of 5000 cells/well in media containing 10% CD-FBS with 10 nM DHT. At the indicated time points, the medium was replenished and cell proliferation was determined by MTT [3-(4,5-dimethylthiazol-2-yl)-2,5-diphenyltetrazolium bromide] assay. Serum-free medium containing MTT (0.5 mg/ml, Sigma) was added into each well and after 4 h incubation at 37° C., cellular formazan product was solubilized with acidic isopropanol, and the absorbance at OD595 was measured by spectrophotometry (Beekman Du640B). 6 replicate wells were used for each sample at each time point.

Stable transfected LNCaP and PC-3 subline cells were grown in 100-mm dishes, digested the cells by trypsin-EDT, harvested as many as 1×10⁶ cells, and fixed them in 70% ethanol at 4° C. After 12 hr, cells were centrifuged (1000 g, 7 min, 4° C.) cells, resuspended in PBS containing 0.05 mg/mL RNase A (Sigma), and then incubated at room temperature for 30 min. After washing, the cells were stained with 10 mg/ml propidium iodide, filtered through a 60 mm mesh, and analyzed 10,000 cells by flow cytometry (FACSCalibur, BD Company) with MODFIT software (Verity Software House, Inc).

7-AAD (Sigma) was dissolved in acetone, diluted in phosphate-buffered saline (PBS) at a concentration of 200 μg/ml, and kept at 20° C. in the dark until use. 100 μl of 7-AAD solution was added to 2.5×10⁶ target cells in 1 ml of PBS and incubated for 20 min at 4° C. protected from light. The cells were washed in Wash buffer and total DNA was stained with 7-amino-actinomycin D (7-AAD; 20 μL per sample), followed by flow cytometric analysis using FACSort (Becton Dickinson). To assess apoptosis 5×10⁵ cells in were resuspended PBS, analyzed by flow cytometry, and determined total DNA content (7-AAD) using Cell Quest (Becton Dickinson) and FCS Express software (De Novo Software).

### (5) Real Time RT-PCR

Cells were harvested in Trizol (Invitrogen). Following extraction, 5 μg total RNA was reversely transcribed into cDNA by the SuperScript III kit (Invitrogen). Real-time PCR was performed on the iCycler IQ multicolor real-time PCR detection system with 1/5 μl cDNA amplified by SYBR Green PCR Master Mix. Primers were designed by Beacon Designer 2 software as follows: NKX3.1 (forward: 5′-ATGGTTCCAGAACAGACGCTAT-3′, reverse: 5′-TGCCCACGCAGTACAGGTAT-3′); PSP94 (forward: 5′-TCCTGGGCAGCGTTGTGA-3′, reverse: 5′-TTGGGTGTTTGTTTCCTTTGAG-3′); PSMA (forward: 5′-AAGGAAGGGTGGAGACCTAG-3′; reverse: 5′-ACTGAACTCTGGGGAAGGAC-3′); p53 (forward: 5′-TACATGTGTTAACAGTTCCTGCA-3′; reverse: 5′-TTCTGACAACGATCGGAGGA-3′); MDM2 (forward: 5′-GCAGGGGAGAGTGATACAGAT-3′, reverse: 5′-GATGGCTGAGAATAGTCTTCA-3′); HoxA5 (forward: 5′-AGCAGCAGAGAGGGGGTTG-3′, reverse: 5′-GGACGCGTGGATCAGAAAA-3′); egr-1 (forward: 5′-GGCAGGGAGTGATGATTTG-3′, reverse: 5′-GCTCAGCTCAGCCCTCTT-3′). β-Actin (forward: 5′-TGTGCCCATCTACGAGGGGTATGC-3′, reverse: 5′-GGTACATGGTGGTGCCGCCAGACA-3′) was used as internal control. Δthreshold (CT) values were calculated by subtracting the control CT value from the corresponding β-Actin CT from each time point. The absence of nonspecific amplification was confirmed products by agarose-gel electrophoresis.

### (6) Western Blot Analysis

Cell lysates were prepared in RIPA buffer, protein samples were separated on SDS-10% PAGE gel, and they were transferred to a polyvinylidene difluoride membrane. After blocking by 5% non-fat milk and 5% BSA in PBST buffer, the membrane was immunoblottcd with the primary antibody, followed by incubation with AP-conjugated second antibody (Santa Cruz). Finally, the membrane was developed by the AP color developing reagents (Bio-RAD). The monoclonal anti-ARA70 antibody was generated and anti-AR, anti-KLK2, anti-cdk2, rabbit anti-cyclinD1, rabbit anti-p21, rabbit anti-PCNA, goat anti-ORC1-1, rabbit anti-RFC, rabbit anti-bax, rabbit anti-MDM2, goat anti-HoxA5, rabbit anti-egr-1, goat anti-NKX3.1, mouse anti-tubulin, and goat anti-βactin was purchased from Santa Cruz. Anti-p53 and anti-bcl2 monoclonal antibodies were obtained from DAKO (Denmark) and Rabbit anti-phospho-p53 (Ser15 and Ser392) from Cell Signaling Technology, Inc.

### (7) Mammalian Two-Hybrid Assay

For the luciferase assay, 400 ng of pG5-LUC reporter gene plasmid, 200 ng of pVP16-ARA70 and 200 ng of pM-KLK2 were transfected into COS-1 cells with Superfect kit (Qiagen). After 24 h's ethanol or 10 nM DHT treatment, the dual luciferase reporter assay system (Promega) was employed to measure luciferase activity.

### (8) Glutathione S-Transferase (GST) Pull-Down Assay

Briefly, the full-length KLK2 was in vitro translated in the presence of [³⁵S] methionine using T7 polymerase and the coupled transcription/translation kit (Promega). We transformed the PGEX-GST-ARA70 plasmid into BL21-CodonPlus Competent cells (Stratagene) to express GST fusion ARA70 protein, harvested, and purified the GST-ARA70 fusion protein with glutathione-beads. The radiolabeled KLK2 protein was incubated for 2 hr with the GST-ARA70 fusion protein attached to glutathione beads in interaction buffer (50 mM HEPES, 100 mM NaCl, 20 mM Tris-Cl/pH 8.0, 0.1% Tween20, 10% glycerol, 1 mM DTT, 0.5 mM PMSF, 1 mM NaF, and 0.4 mM sodium vanadate). The beads were washed with NENT buffer 4 times, resuspended in SDS-PAGE loading buffer, and resolved on 12% SDS-PAGE gel electrophoresis followed by autoradiography.

### c) Results

### (1) KLK2 Increases Cell Growth in LNCaP Cells but not in PC-3 Cells

The endogenous KLK2, ARA70, and AR expression in prostate cell lines were demonstrated in FIG. 7A by RT-PCR and Western blot assay. KLK2 is highly expressed in AR positive LNCaP cells, mildly expressed in CWR22RV1 and not expressed in AR negative PC-3, DU145, and COS-1 cells. To test the KLK2 effects on prostate cancer cell growth, KLK2 cDNA or KLK2-siRNA was stably transfected into high passage (over 50 passage) LNCaP (named LN-KLK2 and LN-siKLK2), and PC-3 cells (named PC-KLK2). MTT assays show that addition of KLK2 results in the increased cell number in AR-positive LNCaP cells, but not in AR-negative PC-3 cells in the presence of 10 nM DHT (FIG. 7B). Further dissection by flow cytometry analysis with propidium iodide staining showed knockdown of endogenous KLK2 in LN-siKLK2 cells induced G1 arrest with the increased G1 phase from 70% to 80% (FIG. 7C upper panel). On the other hand, stable transfection of KLK2 in the LN-KLK2 cells resulted in a G1 phase decreased from 70% to 65% (FIG. 7C upper panel). As a control, addition of KLK2 into PC-3 cells, showed little change on the G1 phase (FIG. 7C lower panel). Using FACS-analysis with 7-AAD staining, it was found that cell apoptosis was increased significantly in LN-siKLK2 cells as compared to control LN-KLK2 cells (21% vs 7%) (FIG. 7D). On the other hand, cell apoptosis was decreased significantly in LN-KLK2 cells as compared to LN-vector cells (7% vs 12%) (FIG. 7D). In contrast, there was little difference in cell apoptosis between PC-KLK2 cells and its control PC-3 cells transfected with backbone vector (data not shown).

### (2) KLK2 Enhances ARA70-Induced AR Transactivation

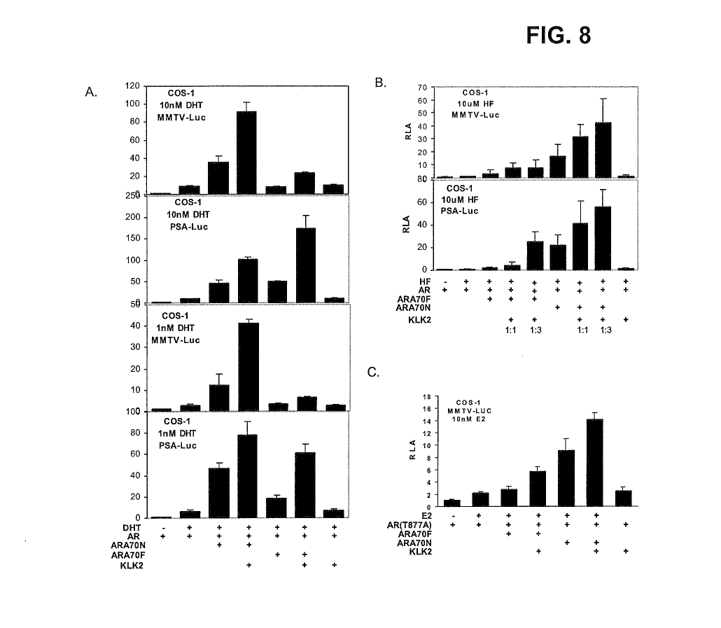

Since KLK2 has effects on AR positive LNCaP calls and has little effect on AR negative PC-3 cells (FIG. 7), whether KLK2 goes through AR signals to increase cell growth was tested. Whether KLK2 can modulate AR activity was tested and it was found that KLK2 could enhance the AR coactivator ARA70-induced AR transactivation in the presence of 10 nM DHT and 1 nM DHT in the green monkey kidney COS-1 cells (FIG. 8A). As early studies suggested that both antiandrogen hydroxyflutamide (HF) and 17β-estradiol (E2) could enhance ARA70-induced AR transactivation (Yeh S, et al., Lancet 1997; 349: 852-853), (Miyamoto H, et al., Proc Natl Acad Sci USA 1998; 95: 7379-7384), the effects of HF and E2 were tested and it was found that KLK2 could also enhance ARA70-induced AR transactivation in the presence of 10 μM HF or 10 nM E2 (FIG. 8B,8C).

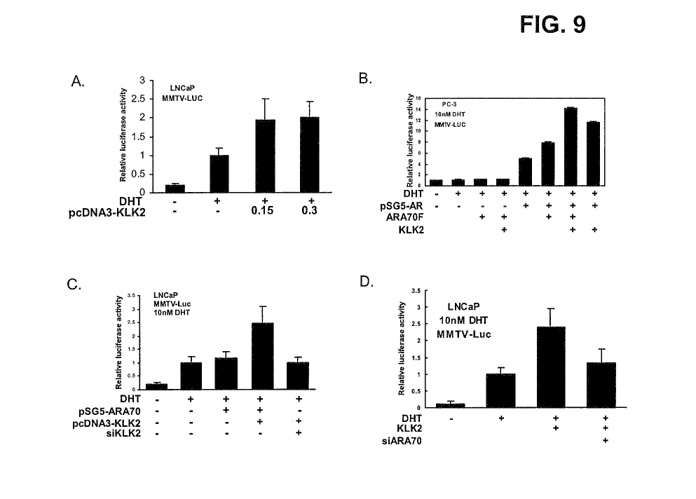

KLK2 could also enhance the ARA70 induced AR transactivation in LNCaP cells (FIG. 9A), as well as in PC-3 cells with stably transfected AR-cDNA (FIG. 9B). Furthermore, when KLK2-cDNA was replaced with KLK2-siRNA that can suppress endogenous KLK2 expression in LNCaP cells, it was found that AR transactivation was reduced in the presence of 10 DHT (FIG. 9C). To test whether this KLK2 effect is through ARA70, the endogenous ARA70 expression in LNCaP cells was knocked down by ARA70 siRNA, and it was found that KLK2 lost such enhanced AR transactivation (FIG. 9D).

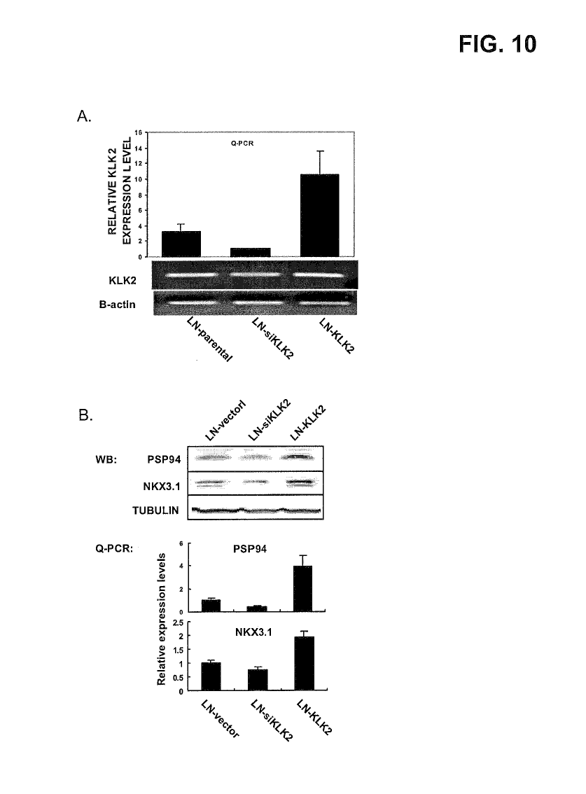

To reduce the potential artificial effects via transient transfection assays, either KLK2-cDNA or KLK2-siRNA were stably transfected into LNCaP cells (FIG. 10A). The KLK2 effects on AR endogenous target genes were examined in LN-KLK2 and LN-siKLK2 cells and the results showed KLK2 could induce AR positive target genes, such as PSP94 (Matusik R J, et al., Biochem Cell Biol 1986; 64: 601-607) and Nkx3.1 (Prescott J L, et al., Prostate 1998; 35:71-80) (FIG. 10B). In contrast, addition of KLK2-siRNA in LN-siKLK2 cells suppressed the expression of these AR target genes (FIG. 10B).

Together, using several cell lines with either transient transfection or stable transfection of KLK2-cDNA or KLK2-siRNA to assay AR transactivation or AR endogenous target gene expression, it was found KLK2 could induce cell growth that might go through the induction of AR trans activation.

### (3) KLK2 Functions as Modulator to Enhance ARA70-Enhanced AR Transactivation

To dissect the molecular mechanisms by which KLK2 can promote ARA70-enhanced AR transactivation, it was found KLK2 could interact with ARA70 in a yeast-two hybrid assay (data not shown). A mammalian two-hybrid system was used to confirm the interaction between KLK2 and ARA70. As shown in FIG. 11A, in the presence or absence of 1 nM DHT, KLK2 could interact relatively well with ARA70, as compared to other AR coactivators (Heinlein C A, et al., Endocr Rev 2002; 23:175-200), such as ARA55, ARA54 and SRC-1 (FIG. 11B). A GST pull-down assay with ARA70 antibody was used to demonstrate that KLK2 could interact with ARA70 (FIG. 11C). Together, data from FIG. 11 using different protein-protein interaction assays (yeast two-hybrid, mammalian two-hybrid, and GST pull-down) all demonstrated that KLK2 could interact with ARA70, and promote ARA70-enhanced AR transactivation.

### (4) Mechanisms by which KLK2 Promoted ARA70-Enhanced AR Transactivation Results in the Increased Prostate Cancer Cell Growth

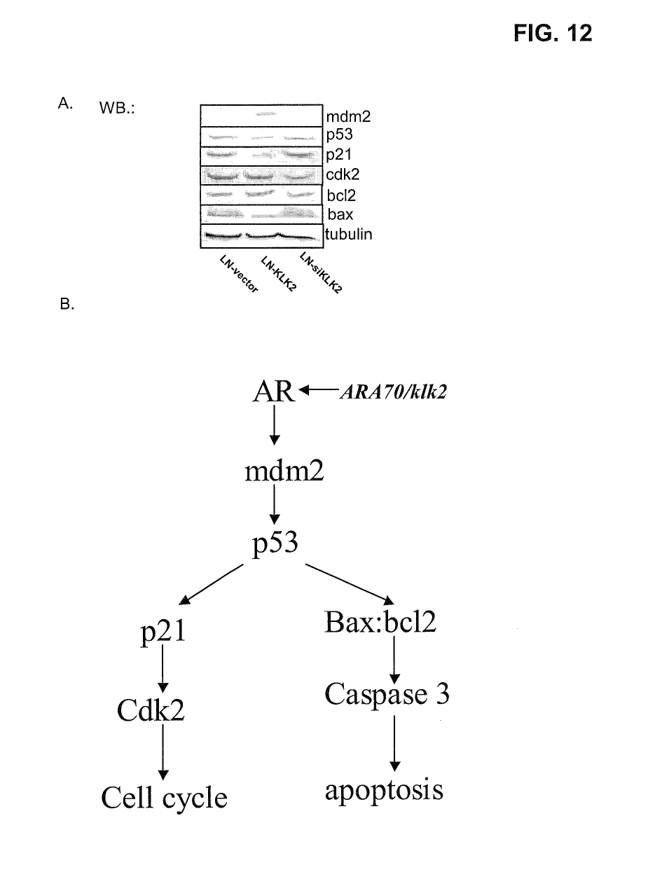

Early studies suggested that androgen/AR might induce cell growth via modulation of p53-mediated cell growth arrest and apoptosis (Ikezoe T, et al., Cancer Sci 2004; 3:271-275), (Hastak K, et al., FASEB. J 2005; 19:789-791). Other studies also demonstrated that several key factors (Nantermet P V, et al., J Biol Chem 2004; 279:1310-1322), such as MDM2, HoxA5, and Egr-I might modulate cell growth arrest and apoptosis via modulation of p53 expression. Whether KLK2 enhanced ARA70-induced AR transactivation could result in increased cell growth via modulation of MDM2→P53 mediated cell growth arrest and apoptosis was tested. Western blot assays were used to evaluate the KLK2 influence on the protein expression of MDM2, p53, and relative signals in LN-KLK2 and LN-siKLK2 cells. As shown in FIG. 12A, higher expression of KLK2 results in the higher expression of MDM2, lower expression of p53, reduced p21 and bax, and increased cdk2 and bcl2 (FIG. 12A). Together, results from FIG. 12 demonstrate that KLK2 can go through the AR-p53 pathway to modulate cell growth and apoptosis.

### d) Discussion

Among the kallikrein family members, proteins expressed by KLK2 and KLK3 (PSA) genes are considered the most closely related KLKs, which can cluster into one subgroup and possess 80% similarity at the amino acid sequence level. For the past 20 years, both of them have been become the important biomarkers for the early diagnosis and management of prostate cancer (Diamandis E P, Trends Endocrinol Metab 1988; 9:310-316), (Clements J A, et al., Crit Rev Clin Lab Sci 2004; 41:265-312). However, the pathophysiological functions remain unclear. Although PSA can modulate the expression of genes involved in prostate tumor growth (Bindukumar B, et al., Neoplasia 2005; 7:241-252), the role of KLK2 remained unknown. Early studies had suggested that the trypsin-like enzyme activity is crucial for KLK2's pathophysiological function (Steuber T, et al., Int J Cancer 2007; 120:1499-1503). Disclosed herein KLK2, can promote prostate cancer cell growth via interaction with the ARA70-AR complex, and therefore represents a new pathophysiological role for KLK2 and indicates that targeting KLK2 itself, can be enough to stop KLK2-induced prostate cancer cell growth.

Western blot analysis was used to simultaneously examine the p53-mediated cell apoptosis markers and it showed lower expression of p53 and bax and higher expression of bcl-2 in LN-KLK2 cells as compared to control LNCaP cells (FIG. 12A). In contrast, higher expression of p53 and bax and lower expression of bcl-2 in LN-siKLK2 cells was observed as compared to control LNCaP cells, Together, these results demonstrated that KLK2 can go through AR signals to modulate p53-induced cell apoptosis. At the hormone refractory stage, both prostate tissue ARA70 and KLK2 expression are higher (Rahman M M, et al., J Biol Chem 2003; 278:19619-19626), (Gregory C W, et al., Cancer Res 1998; 58:5718-5724), (Hu Y C, et al., J Biol Chem 2004; 279, 33438-33446). One possible explanation for the above clinical observations is that with higher expression of ARA70, the androgen ablation therapy with antiandrogen HE might further enhance ARA70-induced AR transactivation, and result in the increased Tissue-KLK2. The increased Tissue-KLK2 can then go through feed-back-positive-regulation to further enhance ARA70-induced AR transactivation that results in the suppression of p53 expression via modulation of MDM2 signal pathways (FIG. 12B). The consequence of AR suppression of p53 can then result in the decrease of cell apoptosis via bax/bcl-2/caspase 3 signal pathways as well as in the decrease of cell G1 arrest via modulation of p21/cdk2 signal pathways (FIG. 12B).

In summary, results disclosed herein showed that KLK2, without involving its protease activity, can promote ARA70-induced AR mediated cell growth. These results not only challenge the classic concept of using KLK2 protease inhibitor to block prostate cancer progression, they also provide a therapeutic target for prostate cancer and AR transactivation. Small molecules that can degrade KLK2 protein or siRNA's for KLK2 or ARA70 to suppress their expression, as well as molecules/peptide(s) to interrupt the interaction between KLK2 and ARA70 can be used.

### e) Reference

Bindukumar B, Schwartz S A, Nair M P N, Aalinkeel R, Kawinski E, and Chadha K C. Prostate-Specific Antigen Modulates the Expression of Genes Involved in Prostate Tumor Growth. Neoplasia 2005; 7:241-252.
C A, M I P, Diamandis E P. Human tissue kallikreins. Physiologic roles and applications in cancer. Mol Cancer Res 2004; 2:257-280.
Clements J A, Willemsen N M, Myers S A, Dong Y. The tissue kallikrein family of serine proteases: functional roles in human disease and potential as clinical biomarkers. Crit Rev Clin Lab Sci 2004; 41:265-312.
Diamandis E P. Prostate-specific antigen: its usefulness in clinical medicine. Trends Endocrinol Metab 1988; 9:310-316.
Diamandis E P, Yousef G M, Luo LY, Magklara A, and Obiezu C V. The new human kallikrein gene family: implications in carcinogenesis. Trend Endocrinol Metab 2000; 11:54-60.
Gregory C W, Hamil K G, Kim D, Hall S H, Pretlow T G, Mohler J L, French F S. Androgen receptor expression in androgen-independent prostate cancer is associated with increased expression of androgen-regulated genes. Cancer Res 1998; 58:5718-5724.
Hastak K, Agarwal M K, Mukhtar H, Agarwal M L. Ablation of either p21 or Bax prevents p53-dependent apoptosis induced by green tea olyphenol epigallocatechin-3-gallate. FASEB. J 2005; 19:789-791.
Heinlein C A, Chang C. Androgen receptor (AR) coregulators: an overview. Endocr Rev 2002; 23:175-200.
Hu Y C, Yeh S, Yeh S D, Sampson E R, Huang J, Li P, Hsu C L, Ting H J, Lin HK, Wang L, Kim E, Ni J, Chang C. Functional domain and motif analyses of androgen receptor coregulator ARA70 and its differential expression in prostate cancer. J Biol Chem 2004; 279, 33438-33446.
Ikezoe T, Yang Y, Saito T, Koeffler H P, and Taguchi H. Proteasome inhibitor PS-341 down-regulates PSA and induces growth arrest and apoptosis of androgen-dependent human prostate cancer LNCaP cells. Cancer Sci 2004; 3:271-275.
Kumar A, Mikolajczyk S D, Goel A S, Millar L S, Saedi M S. Expression of pro form of prostate-specific antigen by mammalian cells and its conversion to mature, active form by kallikrein 2. Cancer Res 1997; 57:3111-3114.
LoEvgren L, Valtonen-Andre C, Marsal K, Lilja H, Lundwall A. Measurement of prostate-specific antigen and human glandular kallikrein 2 in different body fluids. J Androl 1999; 20: 348-355.
Matusik R J, Kreis C, McNicol P, Sweetland R, Mullin C, Fleming, W H, Dodd J G. Regulation of prostatic genes: Role of androgens and zinc in gene expression. Biochem Cell Biol 1986; 64: 601-607.
Miyamoto H, Yeh S, Wilding G, Chang C. Promotion of agonist activity of antiandrogens by the androgen receptor coactivator, ARA70, in human prostate DU145 cells. Proc Natl Acad Sci USA 1998; 95: 7379-7384.
Murtha P, Tindall D J, Young C Y. Androgen induction of a human prostate-specific kallikrein, hKLK2: characterization of an androgen response element in the 5′ promoter region of the gene. Biochemistry 1993; 32:6459-6464.
Nantermet P V, Xu J, Yu Y, Hodor P, Holder D, Adamski S, Gentile M A, Kimmel D B, Harada S, Gerhold D, Freedman L P, and Ray W J. Identification of genetic pathways activated by the androgen receptor during the induction of proliferation in the ventral prostate gland. J Biol Chem 2004; 279:1310-1322.
Prescott J L, Blok L, Tindall D J. Isolation and androgen regulation of the human homeobox cDNA, NKX3.1. Prostate 1998; 35:71-80.
Rahman M M, Miyamoto H, Takatera H, Yeh S, Altuwaijri S, Chang C. Reducing the agonist activity of antiandrogens by a dominant-negative androgen receptor coregulator ARA70 in prostate cancer cells. J Biol Chem 2003; 278:19619-19626.
Solovyeva N I, Balayevskaya T O, Dilakyan E A, Topol L Z, Kisseliov F L. Plasminogen activators, kallikrein-like proteinase and type I and type IV collagenases at various stages of oncogenic transformation. Immunopharmacology 1996; 32:131-134.
Steuber T, Vickers A, Haese A, Kattan M W, Eastham J A, Scardino P T, Huland H, Lilja H. Free PSA isoforms and intact and cleaved forms of urokinase plasminogen activator receptor in serum improve selection of patients for prostate cancer biopsy. Int J Cancer 2007; 120:1499-1503.
Yeh S, Miyamoto H, Chang C. Hydroxyflutamide may not always be a pure antiandrogen. Lancet 1997; 349: 852-853.

### E. SEQUENCES

| 1. SEQ ID NO: 1 ARA70 protein NM_005437 Homo sapiens nuclear | |
|---|---|
| receptor coactivator 4 (NCOA4), mRNA. | |
| MNTFQDQSGSSSNREPLLRCSDARRDLELAIGGVLRAEQQIKDN | |
| LREVKAQIHSCISRHLECLRSREVWLYEQVDLIYQLKEETLQQQAQQLYSLLGQFNCL | |
| THQLECTQNKDLANQVSVCLERLGSLTLKPEDSTVLLFEADTITLRQTITTFGSLKTI | |
| QIPEHLMAHASSANIGPFLEKRGCISMPEQKSASGIVAVPFSEWLLGSKPASGYQAPY | |
| IPSTDPQDWLTQKQTLENSQTSSRACNFFNNVGGNLKGLENWLLKSEKSSYQKCNSHS | |
| TTSSFSIEMEKVGDQELPDQDEMDLSDWLVTPQESHKLRKPENGSRETSEKFKLLFQS | |
| [symbol] LVKTDSCTNCQGNQPKGVEIENLGNLKCLNDHLEAKKPLSTPSMVTEDWLVQ | |
| NHQDPCKVEEVCRANEPCTSFAECVCDENCEKEALYKWLLKKEGKDKNGMPVEPKPEP | |
| EKHKDSLNMWLCPRKEVIEQTKAPKAMTPSRIADSFQVIKNSPLSEWLIRPPYKEGSP | |
| KEVPGTEDRAGKQKFKSPMNTSWCSFNTADWVLPGKKMGNLSQLSSGEDKWLLRKKAQ | |
| EVLLNSPLQEEHNFPPDHYGLPAVCDLFACMQLKVDKEKWLYRTPLQM” | |
| 2. SEQ ID NO: 2 ARA70 nucleotide NM_005437 Homo sapiens nuclear | |
| receptor coactivator 4 (NCOA4), mRNA. | |
| 1 | ctggagttgc cgtgtgacgc gtgggcggga cgaggcccgg gctcggggac ctttcgcact |
| 61 | cgggtcaggg gtaaagcagc ctgtcgcttg ccgggcagct ggtgagtcgg tgacctggcc |
| 121 | tgtgaggagc agtgaggaga atgaatacct tccaagacca gagtggcagc tccagtaata |
| 181 | gagaacccct tttgaggtgt agtgatgcac ggagggactt ggagcttgct attggtggag |
| 241 | ttctccgggc tgaacagcaa attaaagata acttgcgaga ggtcaaagct cagattcaca |
| 301 | gttgcataag ccgtcacctg gaatgtctta gaagccgtga ggtatggctg tatgaacagg |
| 361 | tggaccttat ttatcagctt aaagaggaga cacttcaaca gcaggctcag cagctctact |
| 421 | cgttattggg ccagttcaat tgtcttactc atcaactgga gtgtacccaa aacaaagatc |
| 481 | tagccaatca agtctctgtg tgcctggaga gactgggcag tttgaccctt aagcctgaag |
| 541 | attcaactgt cctgctcttt gaagctgaca caattactct gcgccagacc atcaccacat |
| 601 | ttgggtctct caaaaccatt caaattcctg agcacttgat ggctcatgct agttcagcaa |
| 661 | atattgggcc cttcctggag aagagaggct gtatctccat gccagagcag aagtcagcat |
| 721 | ccggtattgt agctgtccct ttcagcgaat ggctccttgg aagcaaacct gccagtggtt |
| 781 | atcaagctcc ttacataccc agcaccgacc cccaggactg gcttacccaa aagcagacct |
| 841 | tggagaacag tcagacttct tccagagcct gcaatttctt caataatgtc gggggaaacc |
| 901 | taaagggctt agaaaactgg ctcctcaaga gtgaaaaatc aagttatcaa aagtgtaaca |
| 961 | gccattccac tactagttct ttctccattg aaatggaaaa ggttggagat caagagcttc |
| 1021 | ctgatcaaga tgagatggac ctatcagatt ggctagtgac tccccaggaa tcccataagc |
| 1081 | tgcggaagcc tgagaatggc agtcgtgaaa ccagtgagaa gtttaagctc ttattccagt |
| 1141 | cctataatgt gaatgattgg cttgtcaaga ctgactcctg taccaactgt cagggaaacc |
| 1201 | agcccaaagg tgtggagatt gaaaacctgg gcaatctgaa gtgcctgaat gaccacttgg |
| 1261 | aggccaagaa accattgtcc acccccagca tggttacaga ggattggctt gtccagaacc |
| 1321 | atcaggaccc atgtaaggta gaggaggtgt gcagagccaa tgagccctgc acaagctttg |
| 1381 | cagagtgtgt gtgtgatgag aattgtgaga aggaggctct gtataagtgg cttctgaaga |
| 1441 | aagaaggaaa ggataaaaat gggatgcctg tggaacccaa acctgagcct gagaagcata |
| 1501 | aagattccct gaatatgtgg ctctgtccta gaaaagaagt aatagaacaa actaaagcac |
| 1561 | caaaggcaat gactccttct agaattgctg attccttcca agtcataaag aacagcccct |
| 1621 | tgtcggagtg gcttatcagg cccccataca aagaaggaag tcccaaggaa gtgcctggta |
| 1681 | ctgaagacag agctggcaaa cagaagttta aaagccccat gaatacttcc tggtgttcct |
| 1741 | ttaacacagc tgactgggtc ctgccaggaa agaagatggg caacctcagc cagttatctt |
| 1801 | ctggagaaga caagtggctg cttcgaaaga aggcccagga agtattactt aattcacctc |
| 1861 | tacaggagga acataacttc cccccagacc attatggcct ccctgcagtt tgtgatctct |
| 1921 | ttgcctgtat gcagcttaaa gttgataaag agaagtggtt atatcgaact cctctacaga |
| 1981 | tgtgaaggaa tggacaagag ttgagcagcc tttctgctga ttatcacaca tcatgagctg |
| 2041 | agtgactgca gcttgccaaa tctttgtgtt tctgggtctg accaattagc ttagttcttc |
| 2101 | tcctgcctaa ttttgaacta gtaaagcaaa gtgagtcatc agattatgag ttactgttta |
| 2161 | aaagaaaaat gctgtttatt catgctgagg tgattcagtt ccctccttct tacagaagta |
| 2221 | ttttaattca ccccacacta gaaatgcagc atctttgtgg acgtcttttt cacaagcctc |
| 2281 | caaggctcct tagattgggt cgttactaaa agtacattaa aacactcttg tttatcgaag |
| 2341 | tatattgatg tattctaaag ctagtaaact tccctaacgt ttaattgccc tacagatgct |
| 2401 | tctcttgctg tgggttttct tttgttagtg gtctgaaata attattttcc tgttctatta |
| 2461 | atacatagtg tattttgcac aaaaaaatta acctggtcaa tagtgattac caaaatatat |
| 2521 | attaataatc ttggcaattt ttgacattaa ttatgaaaca ttttagccca cgttagttct |
| 2581 | acattattct tcacttaaac tcagctactg caaattttgt ctttctgtaa atgttattaa |
| 2641 | aatatccagt gagctcttta gaaggactca gtattatttc aagactattt ttgaggtaat |
| 2701 | tctagccttt taaaatattc tacagaccta cggggcttaa aagaacccca gtaccgacta |
| 2761 | agcaaatagg caaaagacat gttggaaatg tagtatagta cttgaaacag tcactatcat |
| 2821 | agggataatt ggtgcatcct gtgtaaatgg aagctgagct tgacacctgg tgcttttaag |
| 2881 | tagggataaa gtcatcctct cactgcaagc acagcatacc tgtacctcca aaagtgacgt |
| 2941 | tttagtgaac aggccgtttt caacacttgt gccttggggt gttcattgaa gctttgtgaa |
| 3001 | aactactgat gttttctcag tctccttaaa gttacgtcca tgctttaaaa tgtctgtgta |
| 3061 | ggagagaagt ggggtttata atgttttctc taagatatct ttgctgcttt ccagactttg |
| 3121 | aaactattaa gcttcttaac tgcctcttac cggaaatact tctggggaaa cttcatggtc |
| 3181 | ccaaaatgtc attgccatac agcttcacta gagttctttg aaccacagct gaaaagagct |
| 3241 | ttgtattatt ttttaattcc ctccccagat atcatttagg agtattatat aaaggtggtg |
| [symbol] | [symbol] gtaaggag cctttccagt tatcttgagt tgcagctctg tagtttcttg |
| 3361 | aggccaaaca cactgtattt tacaagtcaa aatataattt acattaatca ctatgttaat |
| 3421 | gagtatgtaa aacattcttt tgcattgatg aattttgtat ctgcttccat taaaagcata |
| 3481 | acagccacaa aaaaaaaaaa aaaaa |
| 3. SEQ ID NO: 3 LOCUS NM_00100223I Protein Homo sapiens | |
| kallikrein-related peptidase 2 (KLK2), transcript variant 2, mRNA | |
| /translation = “MWDLVLSIALSVGCTGAVPLIQSRIVGGWECEKHSQPWQVAVYS | |
| HGWAHCGGVLVHPQWVLTAAHCLKKNSQVWLGRHNLFEPEDTGQRVPVSHSFPHPLYN | |
| MSLLKHQSLRPDEDSSHDLMLLRLSEPAKITDVVKVLGLPTQEPALGTTCYASGWGSI | |
| EPEEFLRPRSLQCVSLHLLSNDMCARAYSEKVTEFMLCAGLWTGGKDTCGVSHPYSQH | |
| LEGKG” | |
| 4. SEQ ID NO: 4 LOCUS NM_001002231 mRNA Homo sapiens kallikrein- | |
| related peptidase 2 (KLK2), transcript variant 2, mRNA | |
| 1 | agccccaaac tcaccacctg gccgtggaca cctgtgtcag catgtgggac ctggttctct |
| 61 | ccatcgcctt gtctgtgggg tgcactggtg ccgtgcccct catccagtct cggattgtgg |
| 121 | gaggctggga gtgtgagaag cattcccaac cctggcaggt ggctgtgtac agtcatggat |
| 181 | gggcacactg tgggggtgtc ctggtgcacc cccagtgggt gctcacagct gcccattgcc |
| 241 | taaagaagaa tagccaggtc tggctgggtc ggcacaacct gtttgagcct gaagacacag |
| 301 | gccagagggt ccctgtcagc cacagcttcc cacacccgct ctacaatatg agccttctga |
| 361 | agcatcaaag ccttagacca gatgaagact ccagccatga cctcatgctg ctccgcctgt |
| 421 | cagagcctgc caagatcaca gatgttgtga aggtcctggg cctgcccacc caggagccag |
| 481 | cactggggac cacctgctac gcctcaggct ggggcagcat cgaaccagag gagttcttgc |
| 541 | gccccaggag tcttcagtgt gtgagcctcc atctcctgtc caatgacatg tgtgctagag |
| 601 | cttactctga gaaggtgaca gagttcatgt tgtgtgctgg gctctggaca ggtggtaaag |
| 661 | acacttgtgg ggtgagtcat ccctactccc aacatctgga ggggaaaggg tgattctggg |
| 721 | ggtccacttg tctgtaatgg tgtgcttcaa ggtatcacat catggggccc tgagccatgt |
| 781 | gccctgcctg aaaagcctgc tgtgtacacc aaggtggtgc attaccggaa gtggatcaag |
| 841 | gacaccatcg cagccaaccc ctgagtgccc ctgtcccacc cctacctcta gtaaatttaa |
| 901 | gtccacctca cgttctggca tcacttggcc tttctggatg ctggacacct gaagcttgga |
| 961 | actcacctgg ccgaagctcg agcctcctga gtcctactga cctgtgcttt ctggtgtgga |
| 1021 | gtccagggct gctaggaaaa ggaatgggca gacacaggtg tatgccaatg tttctgaaat |
| 1081 | gggtataatt tcgtcctctc cttcggaaca ctggctgtct ctgaagactt ctcgctcagt |
| 1141 | ttcagtgagg acacacacaa agacgtgggt gaccatgttg tttgtggggt gcagagatgg |
| 1201 | gaggggtggg gcccaccctg gaagagtgga cagtgacaca aggtggacac tctctacaga |
| 1261 | tcactgagga taagctggag ccacaatgca tgaggcacac acacagcaag gatgacgctg |
| 1321 | taaacatagc ccacgctgtc ctgggggcac tgggaagcct agataaggcc gtgagcagaa |
| 1381 | agaaggggag gatcctccta tgttgttgaa ggagggacta gggggagaaa ctgaaagctg |
| 1441 | attaattaca ggaggtttgt tcaggtcccc caaaccaccg tcagatttga tgatttccta |
| 1501 | gcaggactta cagaaataaa gagctatcat gctgtggttt attatggttt gttacattga |
| 1561 | tgggatacat actgaaatca gcaaacaaaa cagatgtata gattagagtg tggagaaaac |
| 1621 | agaggaaaac ttgcagttac gaagactggc aacttggctt tactaagttt tcagactggc |
| 1681 | aggaagtcaa acctattagg ctgaggacct tgtggagtgt agctgatcca gctgatagag |
| 1741 | gaactagcca ggtgggggcc tttccctttg gatggggggc atatctgaca gttattctct |
| 1801 | ccaagtggag acttacggac agcatataat tctccctgca aggatgtatg ataatatgta |
| 1861 | caaagtaatt ccaactgagg aagctcacct gatccttagt gtccaaggtt tttactgggg |
| 1921 | gtctgtagga cgagtatgga gtacttgaat aattgacctg aagtcctcag acctgaggtt |
| 1981 | ccctagagtt caaacagata cagcatggtc cagagtccca gatgtacaaa aacagggatt |
| 2041 | catcacaaat cccatcttta gcatgaaggg tctggcatgg cccaaggccc caagtatatc |
| 2101 | aaggcacttg ggcagaacat gccaaggaat caaatgtcat ctcccaggag ttattcaagg |
| 2161 | gtgagccctt tacttgggat gtacaggctt tgagcagtgc agggctgctg agtcaacctt |
| 2221 | ttattgtaca ggggatgagg gaaagggaga ggatgaggaa gcccccctgg ggatttggtt |
| 2281 | tggtcttgtg atcaggtggt ctatggggct atccctacaa agaagaatcc agaaataggg |
| 2341 | gcacattgag gaatgatact gagcccaaag agcattcaat cattgtttta tttgccttct |
| 2401 | tttcacacca ttggtgaggg agggattacc accctggggt tatgaagatg gttgaacacc |
| 2461 | ccacacatag caccggagat atgagatcaa cagtttctta gccatagaga ttcacagccc |
| 2521 | agagcaggag gacgctgcac accatgcagg atgacatggg ggatgcgctc gggattggtg |
| 2581 | tgaagaagca aggactgtta gaggcaggct ttatagtaac aagacggtgg ggcaaactct |
| 2641 | gatttccgtg ggggaatgtc atggtcttgc tttactaagt tttgagactg gcaggtagtg |
| 2701 | aaactcatta ggctgagaac cttgtggaat gcagctgacc cagctgatag aggaagtagc |
| 2761 | caggtgggag cctttcccag tgggtgtggg acatatctgg caagattttg tggcactcct |
| 2821 | ggttacagat actggggcag caaataaaac tgaatcttgt tttcagacct taaaaaaaaa |
| [symbol] | [symbol] |
| 5. SEQ ID NO: 5 LOCUS NM_001030047 Protein 1906 bp | |
| mRNA linear DEFINITION Homo sapiens kallikrein-related peptidase 3 | |
| (KLK3), transcript variant 3, mRNA. | |
| MWVPVVFLTLSVTWIGAAPLILSRIVGGWECEKHSQPWQVLVAS | |
| RGRAVCGGVLVHPQWVLTAAHCIRNKSVILLGRHSLFHPEDTGQVFQVSHSFPHPLYD | |
| MSLLKNRFLRPGDDSSHDLMLLRLSEPAELTDAVKVMDLPTQEPALGTTCYASGWGSI | |
| EPEEFLTPKKLQCVDLHVISNDVCAQVHPQKVTKFMLCAGRWTGGKSTCSWVILITEL | |
| TMPALPMVLHGSLVPWRGGV” | |
| 6. SEQ ID NO: 6 LOCUS NM_001030047 nucleotide 1906 bp | |
| mRNA linear DEFINITION Homo sapiens kallikrein-related peptidase 3 | |
| (KLK3), transcript variant 3, mRNA. | |
| 1 | agccccaagc ttaccacctg cacccggaga gctgtgtcac catgtgggtc ccggttgtct |
| 61 | tcctcaccct gtccgtgacg tggattggtg ctgcacccct catcctgtct cggattgtgg |
| 121 | gaggctggga gtgcgagaag cattcccaac cctggcaggt gcttgtggcc tctcgtggca |
| 181 | gggcagtctg cggcggtgtt ctggtgcacc cccagtgggt cctcacagct gcccactgca |
| 241 | tcaggaacaa aagcgtgatc ttgctgggtc ggcacagcct gtttcatcct gaagacacag |
| 301 | gccaggtatt tcaggtcagc cacagcttcc cacacccgct ctacgatatg agcctcctga |
| 361 | agaatcgatt cctcaggcca ggtgatgact ccagccacga cctcatgctg ctccgcctgt |
| 421 | cagagcctgc cgagctcacg gatgctgtga aggtcatgga cctgcccacc caggagccag |
| 481 | cactggggac cacctgctac gcctcaggct ggggcagcat tgaaccagag gagttcttga |
| 541 | ccccaaagaa acttcagtgt gtggacctcc atgttatttc caatgacgtg tgtgcgcaag |
| 601 | ttcaccctca gaaggtgacc aagttcatgc tgtgtgctgg acgctggaca gggggcaaaa |
| 661 | gcacctgctc gtgggtcatt ctgatcaccg aactgaccat gccagccctg ccgatggtcc |
| 721 | tccatggctc cctagtgccc tggagaggag gtgtctagtc agagagtagt cctggaaggt |
| 781 | ggcctctgtg aggagccacg ggggcagcat cctgcagatg gtcctggccc ttgtcccacc |
| 841 | gacctgtcta caaggactgt cctcgtggac cctcccctct gcacaggagc tggaccctga |
| 901 | agtcccttcc ccaccggcca ggactggagc ccctacccct ctgttggaat ccctgcccac |
| 961 | cttcttctgg aagtcggctc tggagacatt tctctcttct tccaaagctg ggaactgcta |
| 1021 | tctgttatct gcctgtccag gtctgaaaga taggattgcc caggcagaaa ctgggactga |
| 1081 | cctatctcac tctctccctg cttttaccct tagggtgatt ctgggggccc acttgtctgt |
| 1141 | aatggtgtgc ttcaaggtat cacgtcatgg ggcagtgaac catgtgccct gcccgaaagg |
| 1201 | ccttccctgt acaccaaggt ggtgcattac cggaagtgga tcaaggacac catcgtggcc |
| 1261 | aacccctgag cacccctatc aaccccctat tgtagtaaac ttggaacctt ggaaatgacc |
| 1321 | aggccaagac tcaagcctcc ccagttctac tgacctttgt ccttaggtgt gaggtccagg |
| 1381 | gttgctagga aaagaaatca gcagacacag gtgtagacca gagtgtttct taaatggtgt |
| 1441 | aattttgtcc tctctgtgtc ctggggaata ctggccatgc ctggagacat atcactcaat |
| 1501 | ttctctgagg acacagatag gatggggtgt ctgtgttatt tgtggggtac agagatgaaa |
| 1561 | gaggggtggg atccacactg agagagtgga gagtgacatg tgctggacac tgtccatgaa |
| 1621 | gcactgagca gaagctggag gcacaacgca ccagacactc acagcaagga tggagctgaa |
| 1681 | aacataaccc actctgtcct ggaggcactg ggaagcctag agaaggctgt gagccaagga |
| 1741 | gggagggtct tcctttggca tgggatgggg atgaagtaag gagagggact ggaccccctg |
| 1801 | gaagctgatt cactatgggg ggaggtgtat tgaagtcctc cagacaaccc tcagatttga |
| 1861 | tgatttccta gtagaactca cagaaataaa gagctgttat actgtg |
| 7. SEQ ID NO: 7 AR protein sequence (Accession No. NM.sub.-000044) | |
| MEVQLGLGRVYPRPPSKTYRGAFQNLFQSVREVIQNPGPRHPEA | |
| ASAAPPGASLLLLQQQQQQQQQQQQQQQQQQQQQQQETSPRQQQQQQGEDGSPQAHRR | |
| GPTGYLVLDEEQQPSQPQSALECHPERGCVPEPGAAVAASKGLPQQLPAPPDEDDSAA | |
| PSTLSLLGPTFPGLSSCSADLKDILSEASTMQLLQQQQQEAVSEGSSSGRAREASGAP | |
| TSSKDNYLGGTSTISDNAKELCKAVSVSMGLGVEALEHLSPGEQLRGDCMYAPLLGVP | |
| PAVRPTPCAPLAECKGSLLDDSAGKSTEDTAEYSPFKGGYTKGLEGESLGCSGSAAAG | |
| SSGTLELPSTLSLYKSGALDEAAAYQSRDYYNFPLALAGPPPPPPPPHPHARIKLENP | |
| LDYGSAWAAAAAQCRYGDLASLHGAGAAGPGSGSPSAAASSSWHTLFTAEEGQLYGPC | |
| GGGGGGGGGGGGGGGGGGGGGGGEAGAVAPYGYTRPPQGLAGQESDFTAPDVWYPGGM | |
| VSRVPYPSPTCVKSEMGPWMDSYSGPYGDMRLETARDHVLPIDYYFPPQKTCLICGDE | |
| ASGCHYGALTCGSCKVFFKRAAEGKQKYLCASRNDCTIDKFRRKNCPSCRLRKCYEAG | |
| MTLGARKLKKLGNLKLQEEGEASSTTSPTEETTQKLTVSHIEGYECQPIFLNVLEAIE | |
| PGVVCAGHDNNQPDSFAALLSSLNELGERQLVHVVKWAKALPGYRNLHVDDQMAVIQY | |
| SWMGLMVFAMGWRSFTNVNSRMLYFAPDLVFNEYRMHKSRMYSQCVRMRHLSQEFGWL | |
| QITPQEFLCMKALLLFSIIPVDGLKNQKFFDELRMNYIKELDRIIACKRKNPTSCSRR | |
| FYQLTKLLDSVQPIARELHQFTFDLLIKSHMVSVDEPEMMAEIISVQVPKILSGKVKP | |
| IYFHTQ | |
| 8. SEQ ID NO: 8 AR cDNA sequence (Accession No. NM.sub.-000044) | |
| 1 | cgagatcccg gggagccagc ttgctgggag agcgggacgg tccggagcaa gcccagaggc |
| 61 | agaggaggcg acagagggaa aaagggccga gctagccgct ccagtgctgt acaggagccg |
| 121 | aagggacgca ccacgccagc cccagcccgg ctccagcgac agccaacgcc tcttgcagcg |
| 181 | cggcggcttc gaagccgccg cccggagctg ccctttcctc ttcggtgaag tttttaaaag |
| 241 | ctgctaaaga ctcggaggaa gcaaggaaag tgcctggtag gactgacggc tgcctttgtc |
| 301 | ctcctcctct ccaccccgcc tccccccacc ctgccttccc cccctccccc gtcttctctc |
| 361 | ccgcagctgc ctcagtcggc tactctcagc caacccccct caccaccctt ctccccaccc |
| 421 | gcccccccgc ccccgtcggc ccagcgctgc cagcccgagt ttgcagagag gtaactccct |
| 481 | ttggctgcga gcgggcgagc tagctgcaca ttgcaaagaa ggctcttagg agccaggcga |
| 541 | ctggggagcg gcttcagcac tgcagccacg acccgcctgg ttaggctgca cgcggagaga |
| 601 | accctctgtt ttcccccact ctctctccac ctcctcctgc cttccccacc ccgagtgcgg |
| 661 | agccagagat caaaagatga aaaggcagtc aggtcttcag tagccaaaaa acaaaacaaa |
| 721 | caaaaacaaa aaagccgaaa taaaagaaaa agataataac tcagttctta tttgcaccta |
| 781 | cttcagtgga cactgaattt ggaaggtgga ggattttgtt tttttctttt aagatctggg |
| 841 | catcttttga atctaccctt caagtattaa gagacagact gtgagcctag cagggcagat |
| 901 | cttgtccacc gtgtgtcttc ttctgcacga gactttgagg ctgtcagagc gctttttgcg |
| 961 | tggttgctcc cgcaagtttc cttctctgga gcttcccgca ggtgggcagc tagctgcagc |
| 1021 | gactaccgca tcatcacagc ctgttgaact cttctgagca agagaagggg aggcggggta |
| 1081 | agggaagtag gtggaagatt cagccaagct caaggatgga agtgcagtta gggctgggaa |
| 1141 | gggtctaccc tcggccgccg tccaagacct accgaggagc tttccagaat ctgttccaga |
| 1201 | gcgtgcgcga agtgatccag aacccgggcc ccaggcaccc agaggccgcg agcgcagcac |
| 1261 | ctcccggcgc cagtttgctg ctgctgcagc agcagcagca gcagcagcag cagcagcagc |
| 1321 | agcagcagca gcagcagcag cagcagcagc agcaagagac tagccccagg cagcagcagc |
| 1381 | agcagcaggg tgaggatggt tctccccaag cccatcgtag aggccccaca ggctacctgg |
| 1441 | tcctggatga ggaacagcaa ccttcacagc cgcagtcggc cctggagtgc caccccgaga |
| 1501 | gaggttgcgt cccagagcct ggagccgccg tggccgccag caaggggctg ccgcagcagc |
| 1561 | tgccagcacc tccggacgag gatgactcag ctgccccatc cacgttgtcc ctgctgggcc |
| 1621 | ccactttccc cggcttaagc agctgctccg ctgaccttaa agacatcctg agcgaggcca |
| 1681 | gcaccatgca actccttcag caacagcagc aggaagcagt atccgaaggc agcagcagcg |
| 1741 | ggagagcgag ggaggcctcg ggggctccca cttcctccaa ggacaattac ttagggggca |
| 1801 | cttcgaccat ttctgacaac gccaaggagt tgtgtaaggc agtgtcggtg tccatgggcc |
| 1861 | tgggtgtgga ggcgttggag catctgagtc caggggaaca gcttcggggg gattgcatgt |
| 1921 | acgccccact tttgggagtt ccacccgctg tgcgtcccac tccttgtgcc ccattggccg |
| 1981 | aatgcaaagg ttctctgcta gacgacagcg caggcaagag cactgaagat actgctgagt |
| 2041 | attccccttt caagggaggt tacaccaaag ggctagaagg cgagagccta ggctgctctg |
| 2101 | gcagcgctgc agcagggagc tccgggacac ttgaactgcc gtctaccctg tctctctaca |
| 2161 | agtccggagc actggacgag gcagctgcgt accagagtcg cgactactac aactttccac |
| 2221 | tggctctggc cggaccgccg ccccctccgc cgcctcccca tccccacgct cgcatcaagc |
| 2281 | tggagaaccc gctggactac ggcagcgcct gggcggctgc ggcggcgcag tgccgctatg |
| 2341 | gggacctggc gagcctgcat ggcgcgggtg cagcgggacc cggttctggg tcaccctcag |
| 2401 | ccgccgcttc ctcatcctgg cacactctct tcacagccga agaaggccag ttgtatggac |
| 2461 | cgtgtggtgg tggtgggggt ggtggcggcg gcggcggcgg cggcggcggc ggcggcggcg |
| 2521 | gcggcggcgg cggcgaggcg ggagctgtag ccccctacgg ctacactcgg ccccctcagg |
| 2581 | ggctggcggg ccaggaaagc gacttcaccg cacctgatgt gtggtaccct ggcggcatgg |
| 2641 | tgagcagagt gccctatccc agtcccactt gtgtcaaaag cgaaatgggc ccctggatgg |
| 2701 | atagctactc cggaccttac ggggacatgc gtttggagac tgccagggac catgttttgc |
| 2761 | ccattgacta ttactttcca ccccagaaga cctgcctgat ctgtggagat gaagcttctg |
| 2821 | ggtgtcacta tggagctctc acatgtggaa gctgcaaggt cttcttcaaa agagccgctg |
| 2881 | aagggaaaca gaagtacctg tgcgccagca gaaatgattg cactattgat aaattccgaa |
| 2941 | ggaaaaattg tccatcttgt cgtcttcgga aatgttatga agcagggatg actctgggag |
| 3001 | cccggaagct gaagaaactt ggtaatctga aactacagga ggaaggagag gcttccagca |
| 3061 | ccaccagccc cactgaggag acaacccaga agctgacagt gtcacacatt gaaggctatg |
| 3121 | aatgtcagcc catctttctg aatgtcctgg aagccattga gccaggtgta gtgtgtgctg |
| 3181 | gacacgacaa caaccagccc gactcctttg cagccttgct ctctagcctc aatgaactgg |
| 3241 | gagagagaca gcttgtacac gtggtcaagt gggccaaggc cttgcctggc ttccgcaact |
| 3301 | tacacgtgga cgaccagatg gctgtcattc agtactcctg gatggggctc atggtgtttg |
| 3361 | ccatgggctg gcgatccttc accaatgtca actccaggat gctctacttc gcccctgatc |
| 3421 | tggttttcaa tgagtaccgc atgcacaagt cccggatgta cagccagtgt gtccgaatga |
| 3481 | ggcacctctc tcaagagttt ggatggctcc aaatcacccc ccaggaattc ctgtgcatga |
| 3541 | aagcactgct actcttcagc attattccag tggatgggct gaaaaatcaa aaattctttg |
| 3601 | atgaacttcg aatgaactac atcaaggaac tcgatcgtat cattgcatgc aaaagaaaaa |
| 3661 | atcccacatc ctgctcaaga cgcttctacc agctcaccaa gctcctggac tccgtgcagc |
| 3721 | ctattgcgag agagctgcat cagttcactt ttgacctgct aatcaagtca cacatggtga |
| 3781 | gcgtggactt tccggaaatg atggcagaga tcatctctgt gcaagtgccc aagatccttt |
| 3841 | ctgggaaagt caagcccatc tatttccaca cccagtgaag cattggaaac cctatttccc |
| 3901 | caccccagct catgccccct ttcagatgtc ttctgcctgt tataactctg cactactcct |
| 3961 | ctgcagtgcc ttggggaatt tcctctattg atgtacagtc tgtcatgaac atgttcctga |
| 4021 | attctatttg ctgggctttt tttttctctt tctctccttt ctttttcttc ttccctccct |
| 4081 | atctaaccct cccatggcac cttcagactt tgcttcccat tgtggctcct atctgtgttt |
| 4141 | tgaatggtgt tgtatgcctt taaatctgtg atgatcctca tatggcccag tgtcaagttg |
| 4201 | tgcttgttta cagcactact ctgtgccagc cacacaaacg tttacttatc ttatgccacg |
| 4261 | ggaagtttag agagctaaga ttatctgggg aaatcaaaac aaaaacaagc aaac |

[symbol]

indicates data missing or illegible when filed

### F. REFERENCES

1. Diamandis, E. P 1988. Prostate-specific antigen: its usefulness in clinical medicine. Trends Endocrinol. Metab. 9: 310-316.
2. Borgono, C. A., Michael, I. P., Diamandis, E. P. 2004. Human tissue kallikreins: Physiologic roles and applications in cancer. Mol, Cancer Res. 2: 257-280.
3. Obiezu C V, Scorilas A, Katsaros D, Massobrio M, Yousef G M, Fracchioli S, Rigault de la Longrais I A, Arisio R, Diamandis E P, Higher human kallikrein gene 4 (KLK4) expression indicate poor prognosis of ovarian cancer patients. Clin Cancer Res., 7: 2380-2386, 2001.
4. Anisowicz A, Sotiropoulou G, Stenman G, Mok S C, Sager R. A novel protease homolog differentially expressed in breast and ovarian cancer. Mol Med., 2: 624-636, 1996.
5. Bhattacharjce A, Richards W G, Staunton J, et al. Classification of human lung carcinomas by mRNA expression profiling reveals distinct adenocarcinoma subclasses. Proc Natl Acad Sci USA, 98: 13700-5, 2001.
6. Iacobuzio-Donahue C A, Ashfaq R, Maitra A, et al. Highly expressed genes in pancreatic ductal adenocarcinomas: a comprehensive characterization and comparison of the transcription profiles obtained from three major technologies. Cancer Res., 63: 8614-8622, 2003.
7. Roman-Gomez J, Jimenez-Velasco A, Agirre X, et al. The normal epithelial cell-specific I (NES1) gene, a candidate tumor suppressor gene on chromosome 19ql3.3-4, is down-regulated by hypermethylation in acute lymphoblastic leukemia. Leukemia, 18: 362-365, 2004.
8. Bok, R. A., Small, E. J. 2002. Bloodborne bimolecular markers in prostate cancer development and progression. Nat. Rev. Cancer. 2: 918-926.
9. Grande M, Carlstrom K, Rozell B L, Eneroth P, Stege R, and Pousette A. Tissue concentrations of tissue polypeptide antigen (TPA) and prostatic specific antigen (PSA) in 42 patients with prostatic carcinoma. Prostate, 45: 299-303, 2000.
10. Cohen, P., Graves, H. C., Peehl, D. M., Kamarei, M., Giudice, L. C., Rosenfeld, R. G. 1992. Prostate-specific antigen (PSA) is an insulin-like growth factor binding protein-3 protease found in seminal plasma. J. Clin. Endocrinol. Metab. 75: 1046-1053.
11. Denmeade, S. R., Litvinov, I., Sokoll, L. J., Lilja, H., Isaacs, J.T. 2003. Prostate-specific antigen (PSA) protein does not affect growth of prostate cancer cells in vitro or prostate cancer xenografts in vivo. Prostate 56: 45-53.
12. Cleutjens K B J M, Van der Korput H A G M, van Eekelen C C E M, van Rooij H C J, Faber P W, Trapman J. An androgen response element in a far upstream enhancer region is essential for high, androgen-regulated activity of the prostate-specific antigen promoter. Mol Endocrinol 1997; 11:148-161.
13. Kollara, A., Diamandis, E. P., and Brown, T. J. 2003. Secretion of endogenous kallikreins 2 and 3 by androgen receptor-transfected PC3 prostate cancer cells. J. Steroid Biochem. Mol. Biol. 84: 493-502.
14. Chen C D, Welsbie D S, Tran C, Back S H, Chen R, Vessella R, et al. Molecular determinants of resistance to antiandrogen therapy. Nature Med 2004; 10: 33-39.
15. Cluetjens C B J M, Steketee K, van Eekelen C C E M, van der Korput J A G M, Brinkmann A O, Trapman J. Both androgen receptor and glucocorticoid receptor are able to induce prostate-specific antigen expression, but differ in their growth stimulating properties of LNCaP cells. Endocrinology 1997; 138:5293-5300, 1997.
16. Yu H, Diamandis E P, Zargbami N, Grass L. Induction of prostate-specific antigen production by steroids and tamoxifen in breast cancer cell lines. Breast Cancer Res Treat 1994; 32: 291-300.
17. de Winter J A R, Janssen P J A, Sleddens H M E B, Verleun-Mooijman M C T, Trapman J, Brinkmann A O, et al. Androgen receptor status in localized and locally progressive hormone refractory human prostate cancer. Am J Pathol 1994; 144: 735-746.
18. Lodish, H., Berk, A., Zipursky, S. L., Matsudaira, P., Baltimore, D., Darnell, J. E. 2000. Molecular Cell Biology, 4^{th} edition, New York, W. H. Freeman and Company, 691-696 pp.
19. Novick, P., Field, C., and Schekman, R. 1980. Identification of 23 Complementation Groups Required for Post-translational Events in the Yeast Secretory Pathway. Cell 21: 205-215.
20. Novick, P., Ferro, S., and Schekman, R. 1981. Order of events in the yeast secretory pathway. Cell 25: 461-469.
21. Kaiser, C. A. and Schekman, R. 1990. Distinct sets of SEC genes govern transport vesicle formation and fusion early in the secretory pathway. Cell 61: 723-733.
22. Schekman, R. 2002. SEC mutants and the secretory apparatus. Nature Medicine 8: 1055-1058.
23. Matusik, R. J., Kreis, C., McNicol, P., Sweetland, R., Mullin, C., Fleming, W. H., Dodd, J. G. 1986. Regulation of prostatic genes: Role of androgens and zinc in gene expression. Biochem. Cell. Biol. 64: 601-607.
24. Prescott, J. L., Blok, L., Tindall, D. J. 1998. Isolation and androgen regulation of the human homeobox cDNA, NKX3.1. Prostate 35: 71-80.
25. Israeli, R. S., Powell, C. T., Fair, W R., Heston, W. D. W 1993. Molecular cloning of a complementary DNA encoding a prostate-specific membrane antigen. Cancer Res. 53: 227-230.
26. Wright, G. L., Jr., Grob, B. M., Haley, C., Grossman, K., Newhall, K., Petrylak, D., Troyer, J., Konchuba, A., Schellhammer, R E, Moriarty, R. 1996. Upregulation of prostate-specific membrane antigen after androgen-deprivation therapy, Urology 48: 326-334.
27. Yeh, S., Miyamoto, H., Chang, C. 1997. Hydroxyflutamide may not always be a pure antiandrogen. Lancet 349: 852-853.
28. Miyamoto, H., Yeh, S., Wilding, G., Chang, C. 1998. Promotion of agonist activity of antiandrogens by the androgen receptor coactivator, ARA70, in human prostate DU145 cells. Proc. Natl. Acad. Sci. USA. 95: 7379-7384.
29. Nelson, P. S., Gan, L., Ferguson, C., Moss, P., Gelinas, R., Hood, L., Wang, K. 1999. Molecular cloning and characterization of prostase, an androgen-regulated serine protease with prostate-restricted expression. Proc. Natl. Acad. Sci USA. 96: 3114-3119.
30. Ikezoe T, Yang Y, Saito T, Koerner H P, and Taguchi H. 2004. Proteasome inhibitor PS-341 down-regulates prostate-specific antigen (PSA) and induces growth arrest and apoptosis of androgen-dependent human prostate cancer LNCaP cells. Cancer Sci. 3: 271-275.
31. Hastak, K., Agarwal, M. K., Mukhtar, H., Agarwal, M. L. 2005. Ablation of either p21 or Bax prevents p53-dependent apoptosis induced by green tea olyphenol epigallocatechin-3-gallate. FASEB. J. 19: 789-791.
32. Sun, C., Shi, Y., Xu, L. L., Nageswararao, C., Davis, L. D., Segawa, T., Dobi, A., McLeod, D. G., Srivastava, S. 2006. Androgen receptor mutation (T877A) promotes prostate cancer cell growth and cell survival. Oncogene 25: 3905-3913.
33. Nantermet, P. V., Xu, 5., Yu, Y., Hodor, P., Holder, D., Adamski, S., Gentile, M. A., Kimmel, D. B., Harada, S., Gerhold, D., Freedman, L. P., and Ray, W. J. 2004. Identification of genetic pathways activated by the androgen receptor during the induction of proliferation in the ventral prostate gland. J. Biol. Chem 279: 1310-1322.
34. Altuwaijri, S., Lin, H. K., Chuang, K. H., Lin, W J., Yeh, S., Hanchett, L. A., Rahman, M. M., Kang, H. Y, Tsai, M. Y, Zhang, Y., Yang, L., Chang, C. 2003. Interruption of nuclear factor kappaB signaling by the androgen receptor facilitates 12-O-tetradecanoylphorbolacetate-induced apoptosis in androgen-sensitive prostate cancer LNCaP cells. Cancer Res. 63: 7106-7112.
35. Harris, S. L., Levine A. J. 2005. The p53 pathway: positive and negative feedback loops. Oncogene 24: 2899-2908.
36. Webber, M. M., Waghray, A., Bello, D. 1995. Prostate-specific antigen, a serine protease, facilitates human prostate cancer cell invasion. Clin. Cancer Res. 1: 1089-1094.
37. Sutkowski, D. M., Goode, R. L., Banial, J., Teater, C., Cohen, P., McNulty, A. M., Hsiung, H. M., Becker, G. W., Neubauer, B. L. 1999. Growth regulation of prostatic stromal cells by prostate-specific antigen. J. Natl. Cancer. Inst. 91: 1663-1669.
38. Veveris-Lowe, T. L., Lawrence, M. G., Collard, R. L., Bui, L., Heringtaon, A. C., Nicol, D. L., Clements, J. A. 2005. Kallikrein 4 (hK4) and prostate-specific antigen (PSA) are associated with the loss of E-cadherin and an epithelial-mesenchymal transition (EMT)-like effect in prostate cancer cells. Endocr. Relat. Cancer 12: 631-643.
39. Fortier, A. H., Nelson, B. J., Grella, D. K., Holaday, J. W. 1999. Antiangiogenic activity of prostate-specific antigen. J. Natl. Cancer Inst. 91: 1635-1640.
40. Stege, R., Grande, M., Carlstrom, K., Tribukait, B., Pousette, A. 2000. Prognostic significance of tissue prostate-specific antigen in endocrine treated prostate carcinomas. Clin. Cancer Res. 6: 160-165.
41. Rahman, M. M., Miyamoto, H., Takatera, H., Yeh, S., Altuweijri, S., Chang, C. 2003. Reducing the agonist activity of antiandrogens by a dominant-negative androgen receptor coregulator ARA70 in prostate cancer cells. J. Biol. Chem. 278: 19619-19626.
42. Gregory, C. W, Hamil, K. G., Kim, D., Hall, S. H., Pretlow, T. G., Mohler, J. L., French, F. S. 1998. Androgen receptor expression in androgen-independent prostate cancer is associated with increased expression of androgen-regulated genes. Cancer Res 58: 5718-5724.
43. Agus D B, Cordon-Cardo C, Fox W, Drobnjak M, Koff A, Golde D W, Schen H I. 1999. Prostate cancer cell cycle regulator: response to androgen withdrawal and development of androgen independence. J. Natl. Cancer Inst. 93: 1867-1876.
44. Vinall R L, Tepper C G, Shi X-B, Xue L A, Gandour-Edwards R. and de Vere White R. W. 2006. The R273H p53 mutation can facilitate the androgen-independent growth of LNCaP by a mechanism that involves H2 relaxin and its cognate receptor LGR7. Oncogene 25: 2082-2093.
45. Cronauer M V, Schulz W A, Burchardt T, Ackermann R and Burchardt M. 2004. Inhibition of p53 function diminishes androgen receptor-mediated signaling in prostate cancer cell lines. Oncogene 23: 3541-3549.
46. Isaacs W B, Carter B S & Ewing C M. 1991. Wild-type p53 suppresses growth of human prostate cancer cells containing mutant p53 alleles. Cancer Research 51: 4716-4720.
47. Srivastava S, Katayose D, Tong Y A, Craig C R, McLeod D G, Moul J W, Cowan K H, Seth P. 1995. Recombinant adenovirus vector expressing wild-type p53 is a potent inhibitor of prostate cancer cell proliferation. Urology 46: 843-848.
48. Koivistol P A and Rantala 1. 1999. Amplification of the androgen receptor gene is associated with P53 mutation in hormone-refractory recurrent prostate cancer. J. Pathol. 187: 237-241.
49. Hu, Y. C., Yeh, S., Yeh, S. D., Sampson, E. R., Huang, J., Li, P., Hsu, C. L., Ting, H. J., Lin, H. K., Wang, L., Kim, E., Ni, J., Chang, C. 2004. Functional domain and moti analyses of androgen receptor coregulator ARA70 and its differential expression in prostate cancer. J. Biol. Chem. 279: 33438-33446.
50. Yeh, S., Chang, C. 1996. Cloning and Characterization of a specific coactivator, ARA70, for the androgen receptor in human prostate cells. Proc. Natl. Acad. Sci. USA 93: 5517-5521.

1. A method of testing a compound comprising
a. adding the compound to a system, wherein the system comprises PSA or KLK2, wherein the system comprises ARA70, and
b. assaying the effect of the compound on PSA interaction with ARA70 or the effect of the compound on KLK2 interaction with ARA70.

2. (canceled)

3. The method of claim 1, further comprising the step of comparing the effect of the compound to a control.

4. (canceled)

5. The method of claim 1, wherein the compound inhibits PSA-ARA70 interaction or the compound inhibits KLK2-ARA70 interaction.

6-8. (canceled)

9. The method of claim 1, wherein system further comprises AR.

10. (canceled)

11. The method of claim 1, wherein the system further comprises a cell.

12. The method of claim 11, wherein the cell is a hormone refractory cell, an AR-positive cell, a CWR22rv1 cell, or a LNCaP cell.

13-18. (canceled)

19. A method for treating cancer comprising administering a composition to a subject wherein the composition inhibits PSA activity or wherein the composition inhibits KLK2 activity, and wherein the activity of PSA or KLK2 increases cell proliferation.

20. The method of claim 19, wherein the composition comprises a PSA inhibitor or a KLK2 inhibitor.

21. The method of claim 20, wherein the composition does not inhibit the protease activity.

22. The method of claim 20, wherein the PSA inhibitor decreases an interaction between PSA and ARA70.

23. The method of claim 20, wherein the KLK2 inhibitor decreases an interaction between KLK2 and ARA70.

24-29. (canceled)

30. The method of claim 19, further comprising the step of determining whether the subject is in need of administration of a PSA or a KLK2 inhibitor.

31-80. (canceled)

81. The method of claim 11, wherein the method further comprises assaying the effect of the compound on PSA increased cell proliferation or KLK2 increased cell proliferation.


SEQUENCE LISTING (8 sequences)

SEQ ID NO: 1 - protein, 614 aa, Homo sapiens
  Unsure (1)..(614): ARA70 -Homo sapiens nuclear receptor coactivator 4 (NCOA4),
MNTFQDQSGSSSNREPLLRCSDARRDLELAIGGVLRAEQQIKDNLREVKAQIHSCISRHLECLRSREVWLYEQVDLIYQLKEETLQQQAQQLYSLLGQFNCLTHQLECTQNKDLANQVSVCLERLGSLTLKPEDSTVLLFEADTITLRQTITTFGSLKTIQIPEHLMAHASSANIGPFLEKRGCISMPEQKSASGIVAVPFSEWLLGSKPASGYQAPYIPSTDPQDWLTQKQTLENSQTSSRACNFFNNVGGNLKGLENWLLKSEKSSYQKCNSHSTTSSFSIEMEKVGDQELPDQDEMDLSDWLVTPQESHKLRKPENGSRETSEKFKLLFQSYNVNDWLVKTDSCTNCQGNQPKGVEIENLGNLKCLNDHLEAKKPLSTPSMVTEDWLVQNHQDPCKVEEVCRANEPCTSFAECVCDENCEKEALYKWLLKKEGKDKNGMPVEPKPEPEKHKDSLNMWLCPRKEVIEQTKAPKAMTPSRIADSFQVIKNSPLSEWLIRPPYKEGSPKEVPGTEDRAGKQKFKSPMNTSWCSFNTADWVLPGKKMGNLSQLSSGEDKWLLRKKAQEVLLNSPLQEEHNFPPDHYGLPAVCDLFACMQLKVDKEKWLYRTPLQM

SEQ ID NO: 2 - DNA, 3505 bp, Homo sapiens
  Unsure (1)..(3505): ARA70 -Homo sapiens nuclear receptor coactivator 4 (NCOA4)
ctggagttgccgtgtgacgcgtgggcgggacgaggcccgggctcggggacctttcgcactcgggtcaggggtaaagcagcctgtcgcttgccgggcagctggtgagtcggtgacctggcctgtgaggagcagtgaggagaatgaataccttccaagaccagagtggcagctccagtaatagagaaccccttttgaggtgtagtgatgcacggagggacttggagcttgctattggtggagttctccgggctgaacagcaaattaaagataacttgcgagaggtcaaagctcagattcacagttgcataagccgtcacctggaatgtcttagaagccgtgaggtatggctgtatgaacaggtggaccttatttatcagcttaaagaggagacacttcaacagcaggctcagcagctctactcgttattgggccagttcaattgtcttactcatcaactggagtgtacccaaaacaaagatctagccaatcaagtctctgtgtgcctggagagactgggcagtttgacccttaagcctgaagattcaactgtcctgctctttgaagctgacacaattactctgcgccagaccatcaccacatttgggtctctcaaaaccattcaaattcctgagcacttgatggctcatgctagttcagcaaatattgggcccttcctggagaagagaggctgtatctccatgccagagcagaagtcagcatccggtattgtagctgtccctttcagcgaatggctccttggaagcaaacctgccagtggttatcaagctccttacatacccagcaccgacccccaggactggcttacccaaaagcagaccttggagaacagtcagacttcttccagagcctgcaatttcttcaataatgtcgggggaaacctaaagggcttagaaaactggctcctcaagagtgaaaaatcaagttatcaaaagtgtaacagccattccactactagttctttctccattgaaatggaaaaggttggagatcaagagcttcctgatcaagatgagatggacctatcagattggctagtgactccccaggaatcccataagctgcggaagcctgagaatggcagtcgtgaaaccagtgagaagtttaagctcttattccagtcctataatgtgaatgattggcttgtcaagactgactcctgtaccaactgtcagggaaaccagcccaaaggtgtggagattgaaaacctgggcaatctgaagtgcctgaatgaccacttggaggccaagaaaccattgtccacccccagcatggttacagaggattggcttgtccagaaccatcaggacccatgtaaggtagaggaggtgtgcagagccaatgagccctgcacaagctttgcagagtgtgtgtgtgatgagaattgtgagaaggaggctctgtataagtggcttctgaagaaagaaggaaaggataaaaatgggatgcctgtggaacccaaacctgagcctgagaagcataaagattccctgaatatgtggctctgtcctagaaaagaagtaatagaacaaactaaagcaccaaaggcaatgactccttctagaattgctgattccttccaagtcataaagaacagccccttgtcggagtggcttatcaggcccccatacaaagaaggaagtcccaaggaagtgcctggtactgaagacagagctggcaaacagaagtttaaaagccccatgaatacttcctggtgttcctttaacacagctgactgggtcctgccaggaaagaagatgggcaacctcagccagttatcttctggagaagacaagtggctgcttcgaaagaaggcccaggaagtattacttaattcacctctacaggaggaacataacttccccccagaccattatggcctccctgcagtttgtgatctctttgcctgtatgcagcttaaagttgataaagagaagtggttatatcgaactcctctacagatgtgaaggaatggacaagagttgagcagcctttctgctgattatcacacatcatgagctgagtgactgcagcttgccaaatctttgtgtttctgggtctgaccaattagcttagttcttctcctgcctaattttgaactagtaaagcaaagtgagtcatcagattatgagttactgtttaaaagaaaaatgctgtttattcatgctgaggtgattcagttccctccttcttacagaagtattttaattcaccccacactagaaatgcagcatctttgtggacgtctttttcacaagcctccaaggctccttagattgggtcgttactaaaagtacattaaaacactcttgtttatcgaagtatattgatgtattctaaagctagtaaacttccctaacgtttaattgccctacagatgcttctcttgctgtgggttttcttttgttagtggtctgaaataattattttcctgttctattaatacatagtgtattttgcacaaaaaaattaacctggtcaatagtgattaccaaaatatatattaataatcttggcaatttttgacattaattatgaaacattttagcccacgttagttctacattattcttcacttaaactcagctactgcaaattttgtctttctgtaaatgttattaaaatatccagtgagctctttagaaggactcagtattatttcaagactatttttgaggtaattctagccttttaaaatattctacagacctacggggcttaaaagaaccccagtaccgactaagcaaataggcaaaagacatgttggaaatgtagtatagtacttgaaacagtcactatcatagggataattggtgcatcctgtgtaaatggaagctgagcttgacacctggtgcttttaagtagggataaagtcatcctctcactgcaagcacagcatacctgtacctccaaaagtgacgttttagtgaacaggccgttttcaacacttgtgccttggggtgttcattgaagctttgtgaaaactactgatgttttctcagtctccttaaagttacgtccatgctttaaaatgtctgtgtaggagagaagtggggtttataatgttttctctaagatatctttgctgctttccagactttgaaactattaagcttcttaactgcctcttaccggaaatacttctggggaaacttcatggtcccaaaatgtcattgccatacagcttcactagagttctttgaaccacagctgaaaagagctttgtattattttttaattccctccccagatatcatttaggagtattatataaaggtggtgggcaaaaacaatgtaaggagcctttccagttatcttgagttgcagctctgtagtttcttgaggccaaacacactgtattttacaagtcaaaatataatttacattaatcactatgttaatgagtatgtaaaacattcttttgcattgatgaattttgtatctgcttccattaaaagcataacagccacaaaaaaaaaaaaaaaaa

SEQ ID NO: 3 - protein, 223 aa, Homo sapiens
  Unsure (1)..(223): Protein Homo sapiens kallikrein-related peptidase 2 (KLK2)
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKG

SEQ ID NO: 4 - DNA, 2892 bp, Homo sapiens
  Unsure (1)..(2892): Homo sapiens kallikrein-related peptidase 2 (KLK2)
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagcaggacttacagaaataaagagctatcatgctgtggtttattatggtttgttacattgatgggatacatactgaaatcagcaaacaaaacagatgtatagattagagtgtggagaaaacagaggaaaacttgcagttacgaagactggcaacttggctttactaagttttcagactggcaggaagtcaaacctattaggctgaggaccttgtggagtgtagctgatccagctgatagaggaactagccaggtgggggcctttccctttggatggggggcatatctgacagttattctctccaagtggagacttacggacagcatataattctccctgcaaggatgtatgataatatgtacaaagtaattccaactgaggaagctcacctgatccttagtgtccaaggtttttactgggggtctgtaggacgagtatggagtacttgaataattgacctgaagtcctcagacctgaggttccctagagttcaaacagatacagcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagggctgctgagtcaaccttttattgtacaggggatgagggaaagggagaggatgaggaagcccccctggggatttggtttggtcttgtgatcaggtggtctatggggctatccctacaaagaagaatccagaaataggggcacattgaggaatgatactgagcccaaagagcattcaatcattgttttatttgccttcttttcacaccattggtgagggagggattaccaccctggggttatgaagatggttgaacaccccacacatagcaccggagatatgagatcaacagtttcttagccatagagattcacagcccagagcaggaggacgctgcacaccatgcaggatgacatgggggatgcgctcgggattggtgtgaagaagcaaggactgttagaggcaggctttatagtaacaagacggtggggcaaactctgatttccgtgggggaatgtcatggtcttgctttactaagttttgagactggcaggtagtgaaactcattaggctgagaaccttgtggaatgcagctgacccagctgatagaggaagtagccaggtgggagcctttcccagtgggtgtgggacatatctggcaagattttgtggcactcctggttacagatactggggcagcaaataaaactgaatcttgttttcagaccttaaaaaaaaaaaaaaaaaaaaa

SEQ ID NO: 5 - protein, 238 aa, Homo sapiens
  Unsure (1)..(238): Homo sapiens kallikrein-related peptidase 3 (KLK3), transcript variant 3,
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 6 - DNA, 1906 bp, Homo sapiens
  Unsure (1)..(1906): Homo sapiens kallikrein-related peptidase 3 (KLK3),
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 7 - protein, 920 aa, Homo sapiens
  Unsure (1)..(920): AR protein sequence
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 8 - DNA, 4314 bp, Homo sapiens
  Unsure (1)..(4314): AR cDNA sequence
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac